(19) United States
(12) Patent Application Publication
(10) Pub. No.: US 2007/0009518 A1
(43) Pub. Date: Jan. 11, 2007

(54) Methods for treating fibrotic conditions

(75) Inventors: Tatiana Novobrantseva, Quincy, MA (US); Shelia Violette, Lexington, MA (US); Victor Kotelianski, Boston, MA (US); Alexander Ibraghimov, Southborough, MA (US)

Correspondence Address: STERNE, KESSLER, GOLDSTEIN & FOX, P.L.L.C., 1100 NEW YORK AVE., N.W., WASHINGTON, DC, 20005, US

(73) Assignee: Biogen Idec Inc., Cambridge, MA (US)

(21) Appl. No.: 11/436,055

(22) Filed: May 18, 2006

Related U.S. Application Data
(60) Provisional application No. 60/741,867, filed on Dec. 5, 2005.
(60) Provisional application No. 60/682,005, filed on May 18, 2005.

(51) Int. Cl. A61K 39/395; A61K 39/00

(52) U.S. Cl. 424/144.1; 424/185.1

(57) ABSTRACT

The present invention is directed to methods for treating fibrosis conditions, such as liver, kidney and lung fibrosis, as well as fibrosis conditions of other tissues of the body. The methods of the invention comprise administering to a patient in need of such treatment a therapeutically effective amount of a B-cell antagonist. Exemplary B-cell antagonists that can be used in the practice of the methods of the invention include antibodies against B-cell surface antigens (e.g., antibodies against CD20), and BAFF antagonists.

### CROSS-REFERENCE TO RELATED APPLICATIONS

This application claims the benefit of U.S. Provisional Patent Appl. No. 60/682,005, filed May 18, 2005, and of U.S. Provisional Patent Appl. No. 60/741,867, filed Dec. 5, 2005. The contents of the aforementioned applications are incorporated by reference herein in their entireties.

### BACKGROUND OF THE INVENTION

1. Field of the Invention

The present invention relates to methods for the treatment of fibrosis or fibrotic conditions. More specifically, the invention relates to the use of B-cell antagonists or depleting agents to treat fibrosis conditions.

2. Related Art

Tissue damage can result from a variety of chronic or acute stimuli, including infections, autoimmune reactions and mechanical injury. The healing process normally involves a phase during which connective tissue replaces parenchymal tissue. (Wynn, Nature Reviews 4:583-594 (2004)). If this process continues unchecked, however, the formation of permanent scar tissue can result and, in some cases, can ultimately lead to organ failure and death.

Fibrosis conditions are pathological conditions that are characterized by the abnormal and/or excessive accumulation of fibrotic material (e.g., extracellular matrix) following tissue damage. Fibrosis conditions include fibroproliferative disorders that are associated with vascular diseases, such as cardiac disease, cerebral disease, and peripheral vascular disease, as well as in all the main tissues and organ systems, including the skin, kidney, lung, gut and liver. (Wynn, Nature Reviews 4:583-594 (2004)). Although fibrosis conditions are a diverse group of pathologies, it is believed that for most fibrosis conditions, the general mechanisms leading to fibrotic tissue accumulation have many elements in common.

Most therapeutic methods for treating fibrosis conditions target the inflammation response which is believed to play a role in the development of fibrosis generally. (Wynn, Nature Reviews 4:583-594 (2004)). Examples of pharmaceutical strategies for treating fibrosis conditions include the use of immunosuppressive drugs, such as corticosteroids, other traditional immunosuppressive or cytotoxic agents and antifibrotics. There nonetheless exists a need in the art for new and more specifically targeted approaches for the treatment of fibrosis conditions.

### SUMMARY OF THE INVENTION

The present invention is related, at least in part, to the surprising discovery that the extent of experimentally-induced fibrosis injury is substantially reduced in mice that are B-cell deficient or are pharmacologically depleted of B-cells, thereby indicating that depletion of B-cells or impairment of B-cell activity in animals is an effective method for treating fibrosis conditions.

Accordingly, the present invention includes methods for treating fibrosis conditions. The methods of the invention comprise administering to a patient in need of treatment of a fibrosis condition a therapeutically effective amount of a B-cell antagonist.

Since fibrosis is believed to occur by a similar biomolecular mechanism regardless of the specific tissue involved, the present invention can be used to treat any fibrosis condition affecting any tissue in a patient. For example, the present invention can be used to treat, reduce or retard fibrosis of lung (pulmonary), kidney (renal), liver (hepatic), skin, vascular, gut and corneal tissue. The present methods can be used to treat fibrosis conditions resulting from any kind of tissue damage including tissue damage resulting from infections, autoimmune reactions, mechanical injury, chemical, diabetes, hypertension, etc. Specific exemplary fibrosis conditions that can be treated using the methods of the invention are described elsewhere herein.

The methods of the present invention may also be used to prevent a fibrosis condition from developing in a patient at risk of developing a fibrosis condition. Patients at risk of developing a fibrosis condition include, e.g., patients that have been exposed to one or more environmental conditions that are known to cause or stimulate scar tissue accumulation in the lungs, kidney or liver. Exemplary environmental conditions include, e.g., smoke exposure, dust exposure, asbestos exposure, excessive alcohol consumption, radiation exposure, exposure to bleomycin, silica, bacteria, viruses, etc. Patients at risk of developing a fibrosis condition, in certain embodiments, also include, e.g., individuals with diabetes, chronic asthma, lupus, scleroderma, rheumatoid arthritis, vascular disease, glaucoma, IgA neuropathy, Alports syndrome, as well as individuals who have undergone lung transplant and/or kidney transplant.

Exemplary B-cell antagonists that can be used in the practice of the methods of the present invention include any molecule or compound (polypeptide, ligand, fusion protein, antibody, small molecule, etc.) that can inhibit or impair the growth, survival, proliferation or function of B-cells (including the secretion of immunoglobulins), or that can cause the death or destruction of B-cells. The B-cell antagonist according to the present invention may, but not necessarily, function to deplete B-cells. Selected preferred embodiments of the instant invention comprise the use of B-cell antagonists that result in the depletion of at least a portion of circulating or other B-cells through antibody dependent cellular cytotoxicity (ADCC), complement dependent cytotoxicity (CDC) or apoptosis. For the purposes of the instant specification such antagonists may be termed B-cell depleting agents.

In certain embodiments of the invention, the B-cell antagonist or depleting agent is an antibody against a B-cell surface antigen. In particularly preferred embodiments, the B-cell antagonist is an antibody against CD20. An example of an anti-CD20 antibody that can be used in the practice of the methods of the invention is rituximab (RITUXAN®).

In other embodiments of the invention, the B-cell antagonist is an antagonist of BAFF or a BAFF receptor (BR3, BCMA, or TACI), which is expressed on B-cells. Those skilled in the art will appreciate that BAFF is a potent survival factor for B-cells as they transfer from the bone marrow to the spleen during which time autoreactive B-cells are particularly susceptible to becoming pathogenic. Thus, using a BAFF antagonist to interrupt interactions between BAFF and BR can downregulate, interfere with or inhibit the generation of potentially autoreactive B-cells. In this respect, useful antagonists may comprise anti-BAFF antibodies (such as belimumab), anti-BR antibodies, small molecules that interact with BAFF or BR, or ligand based polypeptide antagonists. In particularly preferred embodiments the BAFF antagonist is a soluble molecule comprising all or part of the BAFF receptor linked to an immunoglobulin constant region. Specific exemplary polypeptide BAFF antagonists are discussed in more detail below.

The present invention further includes methods that comprise the administration of multiple B-cell antagonists. For example, in certain embodiments, an antibody against CD20 (e.g., rituximab) is administered to a patient along with a BAFF antagonist.

The invention also includes methods that comprise the administration of one or more B-cell antagonists and one or more additional agents that are useful for treating one or more fibrosis conditions. For example, the present invention also encompasses methods that comprise the administration of one or more B-cell antagonists and one or more integrin receptor antagonists. As those skilled in the art will appreciate integrin receptor antagonists may comprise peptides, antibodies, soluble ligands or small molecules that inhibit the function of an integrin or an integrin receptor, e.g., antibodies against αᵥβ₆, αᵥβ₅, αᵥβ₈, α₅β₁, α₁β₁, α₄β₁ (VLA-4), α₄β₇, etc. An exemplary antibody that specifically binds to the α₄β₁ integrin receptor and that can be used in combination with a B-cell antagonist for the treatment of a fibrotic condition in the context of the present invention is natalizumab (Tysabri®).

The present invention also encompasses methods that comprise the administration of one or more B-cell antagonists and one or more TGF-β pathway inhibitor, such as, e.g., a TGF-β ligand antagonist or a TGF-β receptor antagonist (e.g., monoclonal antibodies, soluble TGF-β RII-Fc fusion protein, LAP-Fc fusion protein, TGF-β RI or RII kinase inhibitors, small molecule inhibitors, etc.)

The skilled artisan will be able to readily identify other anti-fibrotic agents that are compatible with the teachings herein.

Other objects, features and advantages of the present invention will be apparent to those skilled in the art from a consideration of the following detailed description of preferred exemplary embodiments thereof.

### BRIEF DESCRIPTION OF THE FIGURES

FIG. 1A shows the B-cell population in the spleen, peritoneal cavity (PC) and liver of an adult mouse. Lymphocytes were isolated and stained with anti-IgD (X-axis) and anti-IgM (Y-axis). Percentages of IgM⁺, IgD⁺cells among lymphocytes are shown in the plots. FIG. 1B shows the expression levels of CD21, CD23 and CD5 in B-cells isolated from spleen, blood, PC and liver. FIG. 1C shows the amount of Annexin V bound to hepatic B-cells and splenic B-cells. FIG. 1D shows the extent of proliferation of intrahepatic B-cells and splenic B-cells and upregulation of CFSE and CD86 (B7.2) in response to various stimuli.

FIG. 2A shows the degree of liver injury, assessed by the release of the hepatocyte-specific enzyme ALT into serum 24 hours after a single CCl₄ dose, in B-cell deficient mice (J_{H}−/−) and in wild-type mice (BALB/c). FIG. 2B shows the histological analysis of liver tissue stained with the collagen specific dye Sirius Red in B-cell deficient mice (J_{H}−/−) and in wild-type mice (BALB/c) one week after the sixth weekly dose of either oil (control) or CCl₄. FIGS. 2C and 2D show the quantification of collagen specific Sirius Red staining (in arbitrary units) in three representative experiments. Experiments 1 and 2 (FIG. 2C) show the extent of interstitial collagen deposition one week after the sixth weekly dose of 3.5 mg/kg CCl₄, and experiment 3 (FIG. 2D) shows the extent of interstitial collagen deposition one week after the sixth weekly dose of 1.75 mg/kg CCl₄. A column of dots represents a series of sections from one animal. Mean values are shown in bars.

FIG. 3 shows the histological analysis of liver sections of B-cell deficient mice (J_{H}−/−) and wild-type mice (BALB/c), 1, 3 and 5 days after a single CCl₄ challenge. Sections were subjected to either apoptotic specific TUNEL staining (top two rows), smooth muscle actin staining (αSMA) (middle two rows), or macrophage specific F4/80 staining (bottom two rows).

FIG. 4A shows the histological analysis of collagen deposition in liver tissue from mice that lack both B-cells and T-cells (RAG2−/−) and wild-type mice following long term CCl₄ treatment. FIG. 4B shows the quantification of interstitial collagen deposition in liver tissue from RAG2−/− mice and wild-type mice following long term CCl₄ treatment.

FIG. 5A shows the quantification of interstitial collagen deposition in liver tissue from mice expressing Epstein-Barr virus derived LMP2a protein and from wild-type mice following 6 weekly treatments of 1.75 mg/kg CCl₄. FIG. 5B shows the quantification of interstitial collagen deposition in liver tissue from mIgM tg mice expressing surface Ig and from wild-type mice following 6 weekly treatments of 1.75 mg/kg CCl₄.

FIG. 6 shows the percent of α-smooth muscle actin in wild-type “B6BWT” (C57BL/6J) and B-cell deficient “B6BKO” (B6.129S2-lgh-6^{tm1Cgn}/J) mice following 28 days of administration of either 60 mg/kg/7d or 100 mg/kg/7d bleomycin.

FIG. 7 shows the immunohistochemical analysis of lung tissue from wild-type (C57BL/6J) and B-cell deficient (B6.129S2-lgh-6^{tm1Cgn}/J) mice following 28 days of administration of either 100 mg/kg/7d bleomycin or saline.

FIG. 8 shows the percent survival of wild-type mice (C57BL/6J, filled squares) and B-cell deficient mice (B6.129S2-lgh-6^{tm1Cgn}/J, open squares) following administration of 100 mg/kg/7d bleomycin over a 28 day period.

FIGS. 9A, 9B, 9C and 9D show the percent of α-smooth muscle actin (FIG. 9A), interstitial fibrosis (FIG. 9B), dialated tubles (FIG. 9C) and healthy tubules (FIG. 9D) in wild-type “B6Bwt” (C57BL/6J) and B-cell deficient “B6Bko” (B6.129S2-lgh-6^{tm1Cgn}/J) mice subjected to unilateral ureteral obstruction (Op) or unoperated (Unop).

FIG. 10 shows the histological analysis of trichrome stained kidney tissue obtained from wild-type (C57BL/6J) and B-cell deficient (B6.129S2-lgh-6^{tm1Cgn}/J) mice subjected to unilateral ureteral obstruction (Operated) or unoperated.

FIG. 11 shows the B-cell count in the lungs of mice treated with, no bleomycin (control), with bleomycin and with bleomycin plus an anti-CD20 monoclonal antibody.

FIG. 12 shows the splenic B-cell count in mice treated with no bleomycin (control), with bleomycin and with bleomycin plus an anti-CD20 monoclonal antibody.

FIG. 13 shows flow cytometry analysis of B-cells isolated from lungs of untreated mice, or from mice 9 days after bleomycin instillation and treated with a B-cell depleting anti-CD20 monoclonal antibody, or PBS.

FIG. 14 shows the quantification of smooth muscle actin immunostaining in liver tissue from mice treated with either the B-cell depleting anti-CD20 antibody, an isotype control antibody, or PBS, following 6 weekly treatments of 1.75 mg/kg CCl₄. Diamonds, squares, triangles and circles represent the results obtained for individual mice treated as indicated.

### DETAILED DESCRIPTION OF THE INVENTION

The present invention is directed to methods for treating ameliorating, reducing or preventing fibrosis or fibrotic conditions. The methods of the invention comprise administering to a patient in need of such treatment a therapeutically effective amount of a B-cell antagonist.

The expression “fibrosis conditions,” as used herein, is intended to mean any condition in which fibrotic tissue, scar tissue, connective tissue, and/or extracellular matrix (ECM) material accumulates on or within one or more organs within the body in response to tissue injury (e.g., infection, autoimmune reaction, mechanical injury, chemical injury, diabetes, hypertension, etc.). As used herein, the expression “fibrosis conditions” and the expression “fibrotic conditions” are intended to have the same meaning.

Exemplary fibrosis conditions include, but are not limited to:

(I) Lung diseases associated with fibrosis, e.g., idiopathic pulmonary fibrosis, radiation induced fibrosis, chronic obstructive pulmonary disease (COPD), scleroderma, bleomycin induced pulmonary fibrosis, chronic asthma, silicosis, asbestos induced pulmonary fibrosis, acute lung injury and acute respiratory distress (including bacterial pneumonia induced, trauma induced, viral pneumonia induced, ventilator induced, non-pulmonary sepsis induced, and aspiration induced);

(II) Chronic nephropathies associated with injury/fibrosis (kidney fibrosis), e.g., lupus, diabetes, scleroderma, glomerular nephritis, focal segmental glomerular sclerosis, IgA nephropathy, hypertension, allograft, Lupus, and Alport;

(III) Gut fibrosis, e.g., scleroderma, and radiation induced gut fibrosis;

(IV) Liver fibrosis, e.g., cirrhosis, alcohol induced liver fibrosis, nonalcoholic steatohepatitis (NASH), biliary duct injury, primary biliary cirrhosis, infection or viral induced liver fibrosis (e.g., chronic HCV infection), and autoimmune hepatitis;

(V) Head and neck fibrosis, e.g., radiation induced;

(VI) Corneal scarring, e.g., LASIX, corneal transplant, and trabeculectomy;

(VII) Hypertrophic scarring and keloids, e.g., burn induced and surgical; and

(VIII) Other fibrotic diseases, e.g., sarcoidosis, scleroderma, spinal cord injury/fibrosis, myelofibrosis, vascular restenosis, atherosclerosis, Wegener's granulomatosis, mixed connective tissue disease, and Peyronie's disease.

The expression “a patient in need of such treatment,” as used herein, is intended to mean a human or non-human animal that is in need of treatment for one or more fibrosis conditions such as, e.g., any of the fibrosis conditions listed above. A “patient in need of such treatment,” may be a human or non-human animal having an accumulation of fibrotic tissue, scar tissue, and/or extracellular matrix material (e.g., collagen, vimentin, actin, etc.) on or within one or more organs within the body. A “patient in need of such treatment” may be, but is not necessarily, a human or non-human animal that has received a clinical diagnosis of one or more fibrosis conditions. A “patient in need of such treatment” may be a human or non-human animal that exhibits one or more symptoms of a fibrosis condition. (Khalil and O'Connor, Canadian Medical Journal 171:153-160 (2004)). For example, a “patient in need of such treatment” may be a human or non-human animal that exhibits one or more symptoms of: a fibrosis condition of the liver (e.g., liver tissue injury or scarring cause by, e.g., viral hepatitis, alcohol abuse, drugs, metabolic diseases due to overload of iron or copper, autoimmune attack of hepatocytes or bile duct epithelium, or congenital abnormalities) (Friedman, J. Biol. Chem. 275:2247-2250 (2000)); a fibrosis condition of the lung (e.g., lung tissue injury or scarring caused by or related to an inflammatory response of the lung to an inciting event, including e.g., idiopathic interstitial pneumonias) (Garantziotis et al., J. Clin. Invest. 114:319-321 (2004)); scleroderma of the skin or other organ(s) (Trojanowska, Frontiers Biosci. 7:d608-618 (2002); and/or a fibrosis condition of the kidney (e.g., kidney tissue injury or scarring related to glomerulosclerosis or tubular interstitial fibrosis) (Negri, J. Nephrol. 17:496-503 (2004)).

According to certain embodiments, the “patient in need of such treatment” does not have and/or is not at risk of having an autoimmune disorder. For example, a “patient in need of such treatment” may be, but is not necessarily, a patient who has not received a clinical diagnosis of one or more autoimmune disorders. A “patient in need of such treatment” may be, but is not necessarily, a patient who does not exhibit one or more symptoms of one or more autoimmune disorders. As used herein, the term “autoimmune disorder” means a non-malignant disease or disorder arising from and directed against an individual's own (self) antigens and/or tissues. (See, e.g., U.S. Patent Appl. Publication No. 2005/0095243.) Thus, in certain exemplary embodiments of the present invention, a “patient in need of such treatment” is a patient who has not received a clinical diagnosis of, or who does not exhibit one or more symptoms of one or more of the following autoimmune disorders: rheumatoid arthritis, juvenile rheumatoid arthritis, systemic lupus erythematosus (SLE), Wegener's disease, inflammatory bowel disease, idiopathic thrombocytopenic purpura (ITP), thrombotic throbocytopenic purpura (TTP), autoimmune thrombocytopenia, multiple sclerosis, psoriasis, IgA nephropathy, IgM polyneuropathies, myasthenia gravis, vasculitis, diabetes mellitus, Reynaud's syndrome, Sjorgen's syndrome or glomerulonephritis.

Non-human animals include, e.g., domestic and farm animals, as well as zoo animals, sports animals and pet animals (e.g., cats, dogs, horses, cows, etc.)

The expression “therapeutically effective amount,” as used herein, refers to an amount of a B-cell antagonist or antagonist which is effective for preventing, ameliorating, treating or improving the symptoms of the fibrosis condition in question. For example, a therapeutically effective amount of a B-cell antagonist, as used herein, may be an amount of a B-cell antagonist sufficient to cause a decrease in one or more markers of a fibrosis condition. Exemplary markers of a fibrosis condition include, e.g., collagen deposition, smooth muscle actin deposition, etc. A therapeutically effective amount of a B-cell antagonist, in certain embodiments of the invention, is an amount of a B-cell antagonist sufficient to cause a 5%, 10%, 15%, 20%, 25%, 30%, 35%, 40%, 45%, 50%, 55%, 60%, 65%, 70%, 75%, 80%, 90%, 95%, or 100% decrease in collagen deposition relative to the level of collagen deposition observed prior to administration of the B-cell antagonist. A therapeutically effective amount of a B-cell antagonist, in certain other embodiments of the invention, is an amount of a B-cell antagonist sufficient to cause a 5%, 10%, 15%, 20%, 25%, 30%, 35%, 40%, 45%, 50%, 55%, 60%, 65%, 70%, 75%, 80%, 90%, 95%, or 100% decrease in smooth muscle actin deposition relative to the level of smooth muscle actin deposition observed prior to administration of the B-cell antagonist. In yet other embodiments, a therapeutically effective amount of a B-cell antagonist is an amount of a B-cell antagonist sufficient to cause a 5%, 10%, 15%, 20%, 25%, 30%, 35%, 40%, 45%, 50%, 55%, 60%, 65%, 70%, 75%, 80%, 90%, 95%, or 100% improvement in organ function (e.g., liver function, lung function, kidney function) relative to organ function observed prior to administration of the B-cell antagonist.

B-Cell Antagonists or Depleting Agents

The expression “B-cell antagonist,” as used herein, is intended to mean any material or agent that inhibits, impairs, retards, ameliorates or downregulates the growth, survival, proliferation or function of B-cells (e.g., by reducing or preventing a humoral response elicited by a B-cell), or that cause the death or destruction of all or part of a population of B-cells. In the case of the latter, such B-cell antagonists may be termed B-cell depleting agents. B-cell antagonists may be synthetic or native-sequence peptides and small molecules that bind to or interact with a B-cell surface antigen or interact with intracellular signaling molecules to inhibit B-cell function. In some embodiments, the B-cell antagonist may be fused to or conjugated with a cytotoxic agent. According to yet other embodiments of the invention, the B-cell antagonist is a fusion protein (e.g., BR-Fc) or antibody, e.g., an antibody against one or more B-cell surface antigens.

As noted above, the B-cell antagonist may be an agent that depletes B-cells upon or after administration of the B-cell antagonist to a patient. For example, the B-cell antagonist may cause a 2% to 100% depletion of B-cells within 24 to 100 hours of administration of the B-cell antagonist.

For example, the B-cell antagonist may cause a 2%, 4%, 6%, 8%, 10%, 12%, 14%, 16%, 18%, 20%, 22%, 24%, 26%, 28%, 30%, 32%, 34%, 36%, 38%, 40%, 42% , 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% depletion of peripheral B-cells within 24 hours of administration of the B-cell antagonist (e.g., as set forth in U.S. Pat. No. 6,399,061).

Alternatively, the B-cell antagonist may cause a 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% depletion of peripheral B-cells within 48 hours of administration of the B-cell antagonist.

In other embodiments, the B-cell antagonist may cause a 2%, 4%, 6%, 8%, 10%, 12%, 14%, 16%, 18%, 20%, 22%, 24%, 26%, 28%, 30%, 32%, 34%, 36%, 38%, 40%, 42%, 44%, 46%, 48%, 50%, 52%, 54%, 56%, 58%, 60%, 62%, 64%, 66%, 68%, 70%, 72%, 74%, 76%, 78%, 80%, 82%, 84%, 86%, 88%, 90%, 92%, 94%, 96%, 98%, or 100% depletion of peripheral B-cells within 72 hours of administration of the B-cell antagonist.

The ability of B-cell antagonists or depleting agents to treat fibrosis conditions may be assayed using one or more in vitro or in vivo fibrosis models. Exemplary fibrosis models include, e.g., trauma-induced fibrosis models (e.g., surgical trauma or organ transplantation, burns, bile-duct occlusion, unilateral ureteral obstruction, ischemia reperfusion, ventilator induced lung injury, vascular balloon injury, nephrectomy, irradiation, traumatic aorto-caval fistula, and rapid ventricular pacing); toxin or drug-induced fibrosis models (e.g., bleomycin, asbestos, silica, ovalbumin, acetaldehyde, carbon tetrachloride, concanavalin A, vinyl chloride, trinitrobenzene sulphonic acid, oxazolone, cyclosporin A, nickel sulfate, and cerulein); autoimmune disease or malfimctioning immune-mediated fibrosis models (e.g., antibody and immune-complex disease models, organ-transplant rejection, tight skin (Tsk)-mouse model, ischaemia-reperfusion injury, graft-versus-host induced, and rheumatoid arthritis); chronic infectious disease-induce fibrosis models (Schistosoma species or chronic viral hepatitis, Aspergillus fumigatus, Mycobacterium tuberculosis, and Trypanosoma cruzi); and genetically engineered mice models or viral infected mice (e.g., transforming growth factor-β (TGF-β) or TGF-β-receptor transgenic and knockout mice, signaling-molecule-deficient mice (e.g., mothers-against-decapentaplegic homologue 3 (SMAD3)-deficient mice), mice deficient for Col4A3 (Alport), mice deficient in molecules that affect TGF-β activation (e.g., α₁-integrin or matrix metalloproteinase 9), and cytokine-gene transgenic, viral infected, and knockout mice (e.g., tumor-necrosis factor, interleukin-4 (IL-4), IL-13 or IL-10)). (Wynn, Nature Reviews 4:583-594). B-cell antagonists of the invention include, e.g., B-cell antagonists that are shown in any of the aforementioned fibrosis models to improve the symptoms of fibrosis or to reduce, retard, impair and/or ameliorate the extent of fibrotic injury or to reduce one or more markers of fibrotic injury. Assaying an agent, including a B-cell antagonist, for its ability to improve the symptoms of fibrosis or to reduce the extent of fibrotic injury in any one of the aforementioned fibrosis models is well within the skill and knowledge of persons of ordinary skill in the art.

Antibodies

The B-cell antagonists or depleting agents of the invention may be an antibody. The term “antibody,” as used herein, includes, e.g., native antibodies, intact monoclonal antibodies, polyclonal antibodies, mutlispecific antibodies (e.g., bispecific antibodies) formed from at least two intact antibodies, antibody fragments (e.g., antibody fragments that bind to and/or recognize one or more antigens), other multivalent antibody constructs, chimeric antibodies, humanized antibodies, human antibodies (Jakobovits et al., Proc. Natl. Acad. Sci. USA 90:2551 (1993); Jakobovits et al., Nature 362:255-258 (1993); Bruggermann et al., Year in Immunol. 7:33 (1993); U.S. Pat. Nos. 5,591,669 and 5,545,807), antibodies and antibody fragments isolated from antibody phage libraries (McCafferty et al., Nature 348:552-554 (1990); Clackson et al., Nature 352:624-628 (1991); Marks et al., J. Mol. Biol. 222:581-597 (1991); Marks et al., Bio/Technology 10:779-783 (1992); Waterhouse et al., Nucl. Acids Res. 21:2265-2266 (1993)). Methods for making and using antibodies are well known in the art. (See, e.g., WO 00/67796 and references cited therein.)

Two antibodies that are particularly useful as B-cell antagonists or depleting agents in conjunction with the present invention are rituximab, which is a murine/human chimeric antibody, and 2H7, a humanized antibody comprising murine CDRs. Rituximab is disclosed in U.S. Pat. No. 6,399,061 while 2H7 and variants thereof are disclosed in WO 04/056312. Each of these documents is incorporated herein by reference in their entirety.

Other anti-CD20 antibodies that are compatible with the teachings herein include the yttrium-[90]-labeled 2B8 murine antibody designated “Y2B8” or “ibritumomab tiuxetan” ZEVALIN®, commercially available from Biogen-Idec (see also U.S. Pat. No. 5,736,137, incorporated herein by reference); murine IgG2a “B1,” also called “tositumomab,” which may be optionally labeled with ¹³¹I to generate the ¹³¹I-B138 antibody (BEXXAR™) (U.S. Pat. No. 5,595,721, incorporated herein by reference); murine monoclonal antibody “1F5” (Press et al., Blood 69:584-591 (1987) and variants thereof including “framework patched” or humanized 1F5 (WO03/002607); ATCC deposit HB-96450); HuMax™-CD20 (a fully human IgG1 antibody, U.S. Patent Appl. Publication No. 2004/167319; WO04/035607, Genmab, Denmark), AME-133 (an optimized CDR grafted antibody, U.S. Patent Appl. Publication No. 2005/025764; WO04/103404, Applied Molecular Evolution), HumaLym™ (a fully human antibody, Intracel), and hA20 (a humanized IgG1 antibody, U.S. Patent Appl. Publication No. 2003/0219433; WO00/74718, Immunomedics); and monoclonal antibodies L27, G28-2, 93-1B3, B-CI or NU-B2 available from the International Leukocyte Typing Workshop (Valentine et al., In: Leukocyte Typing III (McMichael, Ed., p. 440, Oxford University Press (1987)).

The term “antibody fragments,” as used herein, are molecules that comprise a portion of an intact antibody, preferably comprising the antigen-binding or variable region thereof. Examples of antibody fragments include Fab, Fab′, F(ab′)₂, Fv fragments, single-chain Fv (scFv) fragments, domain deleted antibodies, diabodies, linear antibodies, single-chain antibody molecules, and multispecific antibodies formed from antibody fragments.

The term “native antibodies,” as used herein, is intended to mean heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not necessarily evenly distributed throughout the variable domains of antibodies. Variability is generally concentrated in three segments called hypervariable regions both in the light chain and the heavy chain variable domains. The more highly conserved portions of variable domains are called the framework regions (FRs). The variable domains of native heavy and light chains each comprise four FRs, largely adopting a P-sheet configuration, connected by three hypervariable regions, which form loops connecting, and in some cases forming part of, the 0-sheet structure. The hypervariable regions in each chain are held together in close proximity by the FRs and, with the hypervariable regions from the other chain, contribute to the formation of the antigen-binding site of antibodies (Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the V_{H}-V_{L} dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CHI) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known.

The expression “single-chain Fv” or “scFv” antibody fragments, as used herein, is intended to mean antibody fragments that comprise the V_{H} and V_{L} domains of antibody, wherein these domains are present in a single polypeptide chain. Preferably, the Fv polypeptide further comprises a polypeptide linker between the V_{H} and V_{L} domains which enables the scFv to form the desired structure for antigen binding. (Plückthun, The Pharmacology of Monoclonal Antibodies, vol. 113, Rosenburg and Moore eds., Springer-Verlag, New York, pp. 269-315 (1994)).

The term “diabodies,” as used herein, refers to small antibody fragments with two antigen-binding sites, which fragments comprise a heavy-chain variable domain (V_{H}) Connected to a light-chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. (EP 404,097; WO 93/11161; Hollinger et al., Proc. Natl. Acad. Sci. USA, 90:6444-6448 (1993)).

Polyclonal antibodies include antibodies that are raised in animals by multiple subcutaneous (sc) or intraperitoneal (ip) injections of the relevant antigen and an adjuvant. The relevant antigen may be conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂ or R¹N═C═NR, where R and R¹ are different alkyl groups.

To produce polyclonal antibodies, animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with ⅕ to 1/10 the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

The term “monoclonal antibody,” as used herein, refers to an antibody obtained from a population of substantially homogeneous antibodies, e.g., the individual antibodies comprising the population are substantially identical except for possible naturally occurring mutations or minor post-translational variations that may be present. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

The term “monoclonal antibodies,” as used herein, includes “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species (e.g., mouse or rat) or belonging to another antibody class or subclass, as well as fragments of such antibodies, so long as they exhibit the desired biological activity (U.S. Pat. No. 4,816,567; Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences (U.S. Pat. No. 5,693,780).

As used herein, “humanized” forms of non-human (e.g., murine) antibodies refer to chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. (Jones et al., Nature 321:522-525 (1986); Riechmarm et al., Nature 332:323-329 (1988); Verhoeyen et al., Science, 239:1534-1536 (1988); Presta, Curr. Op. Struct. Biol. 2:593-596 (1992); WO 00/67796.)

The term “hypervariable region,” as used herein, refers to the amino acid residues of an antibody which are responsible for antigen-binding. The hypervariable region comprises amino acid residues from a “complementarity determining region” (“CDR”) (e.g., residues 24-34 (L1), 50-56 (L2) and 89-97 (L3) in the light chain variable domain and 31-35 (H1), 50-65 (H2) and 95-102 (H3) in the heavy chain variable domain) (Kabat et al., Sequences of Proteins of Immunological Interest, 5th Ed., Public Health Service, National Institutes of Health, Bethesda, Md. (1991)), and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain). (Chothia and Lesk, J. Mol. Biol. 196:901-917 (1987)). “Framework” or “FR” residues are those variable domain residues other than the hypervariable region residues as herein defined.

B-Cell Surface Antigens

In certain embodiments of the invention, the B-cell antagonists are antibodies against a B-cell surface antigen. As used herein, the expression “B-cell surface antigen” is intended to mean any antigen that is expressed on the surface of B lymphocytes. In some embodiments of the invention, the “B-cell surface antigen” is an antigen that is expressed on the surface of B-cells in healthy individuals. In other embodiments, the “B-cell surface antigen” is an antigen that is expressed on the surface of B-cells of individuals suffering from a disease state. In yet other embodiments, the “B-cell surface antigen” is an antigen that is expressed on the surface of B-cells in both healthy individuals and in individuals suffering from a disease state. According to some embodiments of the invention, the B-cell surface antigen is expressed on B-cells to a greater extent (e.g., 2× greater, 3× greater, 4× greater, 5× greater, 10×greater, 100× greater, or more) than on non-B-cells. Alternatively, the B-cell surface antigen, according to certain embodiments, may be expressed on B-cells to the same extent or to a lesser extent than on non-B-cells. Certain B-cell surface antigens may be constitutively expressed on non-B-cells and/or expressed on activated B-cells. In certain embodiments of the invention, the B-cell surface antigen is expressed only on B-cells.

Exemplary B-cell surface antigens include the CD10, CD19, CD20, CD21, CD22, CD23, CD24, CD37, CD40, CD52, CD53, CD72, CD73, CD74, CDw75, CDw76, CD77, CDw78, CD79a, CD79b, CD80, CD81, CD82, CD83, CDw84, CD85 and CD86 leukocyte surface markers. Other exemplary B-cell surface antigens include toll-like receptors (e.g., TLR-7 and TLR-9), chemokine receptors (e.g., CXCR3), and APRIL (Medema et al., Cell Death Differ. 10:1121-1125 (2003)). The BAFF receptors (BAFFR/BR3, BCMA and TACI) may also be considered B-cell surface antigens for the purposes of the instant disclosure.

According to certain embodiments of the invention, the B-cell surface antigen is CD19. The “CD19” antigen refers to a ˜90 kDa antigen identified, for example, by the HD237-CD19 or B4 antibody (Kiesel et al., Leukemia Research II 12:1119 (1987)). CD19 is found on cells throughout differentiation of the lineage from the stem cell stage up to a point just prior to terminal differentiation into plasma cells. Binding of B-cell surface antigen to CD 19 may cause internalization of the CD 19 antigen.

The B-cell antagonist may be, e.g., Lym-1, an IgG2a antibody which recognizes B-cells; B2, an antibody directed against the CD21 antigen; B3, an antibody directed against the CD22 antigen; or J5, an antibody directed against the CD10 antigen (U.S. Pat. No. 5,843,398). Anti-CD22 antibodies that are useful as B-cell antagonists in the context of the present invention are described, e.g., in U.S. Pat. Nos. 5,484,892, 5,789,557, and 5,789,554, WO 98/42378, WO 00/20864, and WO 98/41641, and in Campana, D. et al, J. Immunol. 134:1524 (1985), Dorken et al, J. Immunol. 150:4719 (1993) and Engel et al, J. Immunol. 150:4519 (1993). In this regard, the anti-CD22 antibody epratuzumab is particularly useful in the present invention.

Additional exemplary CD22 antibodies that can be used as B-cell antagonists in the context of the present invention are described, e.g., in U.S. Pat. Nos. 5,484,892, 5,789,557 and 6,846,476, and in WO98/42378, WO00/20864, and WO98/41641.

According to certain embodiments of the invention, the B-cell surface antigen is CD23. CD23 is a low affinity receptor for IgE. CD23 is known to mediate cell adhesion, regulate IgE and histamine release, rescue B-cells from apoptosis and regulate myeloid cell growth. See, e.g., Conrad, Annu Rev Immunol 8:623-645 (1990); Delespesse et al., Adv. Immunol. 49:149-191 (1991); Bonnefoy et al., Curr Opin Immunol 5:944-947 (1993). Antibodies specific for CD23 and uses thereof are discussed in, e.g., Rector et al., Immunol. 55:481-488 (1985); Suemura et al., J. Immunol. 137:1214-1220 (1986); Noro et al., J. Immunol. 137:1258-1263 (1986); Bonnefoy et al., J. Immunol. 138:2970-2978 (1987); Flores-Romo et al., Science 261:1038-1046 (1993); Sherr et al., J. Immunol. 142:481-489 (1989); Pene et al., Proc. Natl. Acad. Sci., USA 85:6880-6884 (1988); Bonnefoy et al. (WO87/07302); Bonnefoy et al. (WO96/12741)); Bonnefoy et al., Eur. J. Immunol. 20:139-144 (1990); Sarfati et al., J. Immunol. 141:2195-2199 (1988) and Wakai et al., Hybridoma 12:25-43 (1993); See also U.S. Pat. Nos. 7,008,623; 6,893,638; and 6,011,138.

According to certain embodiments of the invention, the B-cell surface antigen is CD80. CD80 (also known as “B7.1”) has been shown to be critical in the generation of immune responses. (Azuma et al., J. Exp. Med. 177:845-850 (1993); Freeman et al., J. Immunol. 143:2714-2722 (1989); Hathcock et al., Science 262:905-911 (1993); Hart et al., Immunol. 79:616-620 (1993)). Antibodies specific for CD80 have been described, including, e.g., a primatized IgG₁ antibody specific to human CD80 designated “IDEC-114.” (U.S. Pat. Nos. 5,736,137; 6,113,898).

According to certain preferred embodiments of the invention, the B-cell surface antigen is CD20. The “CD20” antigen is a ˜35 kDa, non-glycosylated phosphoprotein found on the surface of greater than 90% of B-cells from peripheral blood or lymphoid organs. CD20 is expressed during early pre-B-cell development and remains until plasma cell differentiation. CD20 is present on both normal B-cells as well as malignant B-cells. Other names for CD20 in the literature include “B-lymphocyte-restricted antigen” and “Bp35”. The CD20 antigen is described in Clark et al., Proc. Natl. Acad. Sci. 82:1766 (1985), for example.

Antibodies Against CD20

The B-cell antagonist of the invention may be an antibody against CD20. Any antibody against CD20 known in the art that functions as a B-cell antagonist may be used in the context of the present invention. (See, e.g., U.S. Pat. Nos. 6,682,734, 6,538,124, 6,528,624, 6,455,043, 6,410,391, 6,399,061, 6,368,596, 6,287,537, 6,242,195, 6,224,866, 6,171,586, 6,194,551, 6,120,767, 6,015,542, 6,090,365, 5,849,898, 5,843,439, 5,843,398, 5,776,456, 5,736,137, 5,721,108, 5,677,180, 5,595,721, 5,500,362, 4,861,579; U.S. Patent Appl. Publication Nos. 2005/0069545, 2005/0053602, 2005/0025764, 2004/0167319, 2004/0093621, 2003/0219433, 2003/0157108, 2003/0147885, 2003/01339301, 2003/0103971, 2003/0095963, 2003/0082172, 2003/0068664, 2003/0026801, 2003/0021781, 2002/0197256, 2002/0197255, 2002/0128448, 2002/0058029, 2002/0041847, 2002/0012665, 2002/0009444, 2002/0006404, 2002/0004587; International Patent Appl. Publication Nos. WO00/09160, WO00/27428, WO00/27433, WO00/44788, WO01/10462, WO01/10461, WO01/10460, WO02/04021, WO01/74388, WO01/80884, WO01/97858, WO02/34790, WO02/060955, WO2/096948, WO02/079255, WO98/56418, WO98/58964, WO99/22764, WO99/51642, WO00/42072, WO00/67796, WO01/03734, WO01/77342, WO00/20864, WO01/13945, WO00/67795, WO00/74718, WO00/76542, WO01/72333, WO02/102312, WO03/002607, WO049694, WO03/061694, WO95/03770; and European Patent Appl. Nos. 330,191 and 332,865.)

Exemplary antibodies against CD20 are set forth, e.g., in U.S. Patent Appl. Publication No. 2005/0053602, and include: “C2B8” which is also called “rituximab” (“RITUXAN®”) (U.S. Pat. No. 5,736,137); the yttrium-[90]-labeled 2B8 murine antibody designated “Y2B8” or “ibritumomab tiuxetan” ZEVALIN® (U.S. Pat. No. 5,736,137); murine IgG2a “B1,” also called “tositumomab,” optionally labeled with ¹³¹I to generate the “¹³¹I-B1” antibody (iodine I131 tositumomab, BEXXAR™) (U.S. Pat. No. 5,595,721); murine monoclonal antibody 1“F5” (Press et al., Blood 69:584-591 (1987)) and “framework patched” or humanized 1F5 (WO03/002607); ATCC deposit HB-96450; murine 2H7 and chimeric 2H7 antibody (U.S. Pat. No. 5,677,180); humanized 2H7; huMax-CD20 (Genmab, Denmark); AME-133 (Applied Molecular Evolution); and monoclonal antibodies L27, G28-2, 93-1B3, B-C1 or NU-B2 available from the International Leukocyte Typing Workshop (Valentine et al., In: “Leukocyte Typing III,” McMichael, Ed., p. 440, Oxford University Press (1987)).

The terms “rituximab” or “RITUXAN®” herein refer to the genetically engineered chimeric murine/human monoclonal antibody directed against the CD20 antigen and designated “C2B8” in U.S. Pat. No. 5,736,137, including fragments thereof which retain the ability to bind CD20.

In certain embodiments, the anti-CD20 antibodies bind human and primate CD20. In specific embodiments, the antibodies that bind CD20 are humanized or chimeric CD20 binding antibodies include rituximab (RITUXAN®), m2H7 (murine 2H7), hu2H7 (humanized 2H7) and all its functional variants, including without limitation, hu2H7.v16 (v stands for version), v31, v73, v75, v114, v511, as well as fucose deficient variants. The sequences of some of the hu2H7 variant antibodies are set forth in WO04/056312, which is incorporated by reference herein in its entirety, and are provided below, with the N-terminal amino acid sequence in bold being the leader sequence which is removed in the mature polypeptide:

| | |
|---|---|
| hu2H7.v16 L chain [232 aa]: | |
| MGWSCIILFLVATATGVHSDIQMTQSPSSLSASVGDR | (SEQ ID NO:1) |
| VTITCRASSSVSYMHWYQQKPGKAPKPLIYAPSNLAS | |
| GVPSRFSGSGSGTDFTLTISSLQPEDFATYYCQQWSF | |
| NPPTFGQGTKVEIKRTVAAPSVFIFPPSDEQLKSGTA | |
| SVVCLLNNFYPREAKVQWKVDNALQSGNSQESVTEQD | |
| SKDSTYSLSSTLTLSKADYEKHKVYACEVTHQGLSSP | |
| VTKSFNRGEC | |
| hu2H7.v16 H chain [471 aa]: | |
| MGWSCIILFLVATATGVHSEVQLVESGGGLVQPGGSL | (SEQ ID NO:2) |
| RLSCAASGYTFTSYNMHWVRQAPGKGLEWVGAIYPGN | |
| GDTSYNQKFKGRFTISVDKSKNTLYLQMNSLRAEDTA | |
| VYYCARVVYYSNSYWYFDVWGQGTLVTVSSASTKGPS | |
| VFPLAPSSKSTSGGTAALGCLVKDYFPEPVTVSWNSG | |
| ALTSGVHTFPAVLQSSGLYSLSSVVTVPSSSLGTQTY | |
| ICNVNHKPSNTKVDKKVEPKSCDKTHTCPPCPAPELL | |
| GGPSVFLFPPKPKDTLMISRTPEVTCVVVDVSHEDPE | |
| VKFNWYVDGVEVHNAKTKPREEQYNSTYRVVSVLTVL | |
| HQDWLNGKEYKCKVSNKALPAPIERTISKAKGQPREP | |
| QVYTLPPSREEMTKNQVSLTCLVKGFYPSDIAVEWES | |
| NGQPENNYKTTPPVLDSDGSFFLYSKLTVDKSRWQQG | |
| NVFSCSVMHEALHNHYTQKSLSLSPGK | |
| hu2H7.v31 H chain [471 aa]: | |
| MGWSCIILFLVATATGVHSEVQLVESGGGLVQPGGSL | (SEQ ID NO:3) |
| RLSCAASGYTFTSYNMHWVRQAPGKGLEWVGAIYPGN | |
| GDTSYNQKFKGRFTISVDKSKNTLYLQMNSLRAEDTA | |
| VYYCARVVYYSNSYWYFDVWGQGTLVTVSSASTKGPS | |
| VFPLAPSSKSTSGGTAALGCLVKDYFPEPVTVSWNSG | |
| ALTSGVHTFPAVLQSSGLYSLSSVVTVPSSSLGTQTY | |
| ICNVNHKPSNTKVDKKVEPKSCDKTHTCPPCPAPELL | |
| GGPSVFLFPPKPKDTLMISRTPEVTCVVVDVSHEDPE | |
| VKFNWYVDGVEVHNAKTKPREEQYNATYRVVSVLTVL | |
| HQDWLNGKEYKCKVSNKALPAPIAATISKAKGQPREP | |
| QVYTLPPSREEMTKNQVSLTCLVKGFYPSDIAVEWES | |
| NGQPENNYKTTPPVLDSDGSFFLYSKLTVDKSRWQQG | |
| NVFSCSVMHEALHNHYTQKSLSLSPGK | |

The L chain of v31 is the same as that of v16 above, i.e., SEQ ID NO. 1.

The term “humanized 2H7v.16,” as used herein, refers to an intact antibody or antibody fragment comprising the variable light chain sequence:

| | |
|---|---|
| DIQMTQSPSSLSASVGDRVTITCRASSSVSYMIHWYQ | (SEQ ID NO:4) |
| QKPGKAPKPLIYAPSNLASGVPSRFSGSGSGTDFTLT | |
| ISSLQPEDFATYYCQQWSFMPTFGQGTKVEIKR; | |

and variable heavy sequence:

| | |
|---|---|
| EVQLVESGGGLVQPGGSLRLSCAASGYTFTSYNMHWV | (SEQ ID NO:5) |
| RQAPGKGLEWVGAIYPGNGDTSYNQKFKGRFTISVDK | |
| SKNTLYLQMNSLRAEDTAVYYCARVVYYSNSYWYFDV | |
| WGQGTLVTVSS. | |

Where the humanized 2H7v.16 antibody is an intact antibody, preferably it comprises the v16 light chain amino acid sequence:

| | |
|---|---|
| DIQMTQSPSSLSASVGDRVTITCRASSSVSYMHWYQQ | (SEQ ID NO:6) |
| KPGKAPKPLIYAPSNLASGVPSRFSGSGSGTDFTLTI | |
| SSLQPEDFATYYCQQWSFNPPTFGQGTKVEIKRTVAA | |
| PSVFIFPPSDEQLKSGTASVVCLLNNFYPREAKVQWK | |
| VDNALQSGNSQESVTEQDSKDSTYSLSSTLTLSKADY | |
| EKHKVYACEVTHQGLSSPVTKSFNRGEC; | |

and v16 heavy chain amino acid sequence:

| | |
|---|---|
| EVQLVESGGGLVQPGGSLRLSCAASGYTFTSYNMFIW | (SEQ ID NO:7) |
| VRQAPGKGLEWVGAIYPGNGDTSYNQKFKGRFTISVD | |
| KSKNTLYLQMNSLRAEDTAVYYCARVVYYSNSYWYFD | |
| VWGQGTLVTVSSASTKGPSVFPLAPSSKSTSGGTAAL | |
| GCLVKDYFPEPVTVSWNSGALTSGVHTFPAVLQSSGL | |
| YSLSSVVTVPSSSLGTQTYICNVNHKPSNTKVDKIKV | |
| EPKSCDKTHTCPPCPAPELLGGPSVFLFPPKPKDTLM | |
| ISRTPEVTCVVVDVSHEDPEVKFNWYVDGVEVHNAKT | |
| KPREEQYNSTYRVVSVLTVLHQDWLNGKEYKCKVSNK | |
| ALPAPIEKTISKAKGQPREPQVYTLPPSREEMTKNQV | |
| SLTCLVKGFYPSDIAVEWESNGQPENNYKTTPPVLDS | |
| DGSFFLYSKLTVDKSRWQQGNVFSCSVMIHEALHNHY | |
| TQKSLSLSPGK. | |

Exemplary humanized 2H7 antibody variants comprising the amino acid sequences of v16, except at certain indicated positions of amino acid substitutions, are summarized in Table 1, below. Unless otherwise indicated, the 2H7 variants will have the same light chain as that of v16.

TABLE 1

| 2H7 | Heavy chain (V_{H}) | Light chain (V_{L}) | |
|---|---|---|---|
| version | changes | changes | Fc changes |
| 31 | — | — | S298A, E333A, |
| | | | K334A |
| 73 | N100A | M32L | |
| 75 | N100A | M32L | S298A, E333A, |
| | | | K334A |
| 96 | D56A, N100A | S92A | |
| 114 | D56A, N100A | M32L, S92A | S298A, E333A, |
| | | | K334A |
| 115 | D56A, N100A | M32L, S92A | S298A, E333A, |
| | | | K334A, E356D, |
| | | | M358L |
| 116 | D56A, N100A | M32L, S92A | S298A, K334A, |
| | | | K322A |
| 138 | D56A, N100A | M32L, S92A | S298A, E333A, |
| | | | K334A, K326A |
| 477 | D56A, N100A | M32L, S92A | S298A, E333A, |
| | | | K334A, K326A, |
| | | | N434W |
| 375 | — | — | K334L |
| 588 | — | | S298A, E333A, |
| | | | K334A, K326A |
| 511 | D56A, N100Y, | M32L, S92A | S298A, E333A, |
| | S100aR | | K334A, K326A |

A variant of the preceding humanized 2H7 mAb is 2H7v.31 having the same L chain sequence as SEQ ID NO:6 above, with the H chain amino acid sequence:

| | |
|---|---|
| EVQLVESGGGLVQPGGSLRLSCAASGYTFTSYNMHWV | (SEQ ID NO:8) |
| RQAPGKGLEWVGAIYPGNGDTSYNQKFKGRFTISVDK | |
| SKNTLYLQMNSLRAEDTAVYYCARVVYYSNSYWYFDV | |
| WGQGTLVTVSSASTKGPSVFPLAPSSKSTSGGTAALG | |
| CLVKDYFPEPVTVSWNSGALTSGVHTFPAVLQSSGLY | |
| SLSSVVTVPSSSLGTQTYICNVNHKPSNTKVDKKVEP | |
| KSCDKTHTCPPCPAPELLGGPSVFLFPPKPKDTLMIS | |
| RTPEVTCVVVDVSHEDPEVKFNWYVDGVEVHNAKTKP | |
| REEQYNATYRVVSVLTVLHQDWLNGKEYKCKVSNKAL | |
| PAPIAATISKAKGQPREPQVYTLPPSREEMTKNQVSL | |
| TCLVKGFYPSDIAVEWESNGQPENNYKTTPPVLDSDG | |
| SFFLYSKLTVDKSRWQQGNVFSCSVMHEALHNHYTQK | |
| SLSLSPGK | |

The murine anti-human CD20 antibody, m2H7, has the V_{H} sequence:

| | |
|---|---|
| QAYLQQSGAELVRPGASVKMSCKASGYTFTSYNMHWV | (SEQ ID NO:9) |
| KQTPRQGLEWIGAIYPGNGDTSYNQKFKGKATLTVDK | |
| SSSTAYMQLSSLTSEDSAVYFCARVVYYSNSYWYFDV | |
| WGTGTTVTVS; | |

and VL sequence:

| | |
|---|---|
| QIVLSQSPAILSASPGEKVTMTCRASSSVSYMIHWY | (SEQ ID NO:10) |
| QQKPGSSPKPWIYAPSNLASGVPARFSGSGSGTSYS | |
| LTISRVEAEDAATYYCQQWSFNPPTFGAGTKILELK | |
| . | |

Another preferred humanized 2H7 antibody comprises 2H7.v511 variable light-domain sequence:

| | |
|---|---|
| DIQMTQSPSSLSASVGDRVTITCRASSSVSYLHWY | (SEQ ID NO:11) |
| QQKIPGKAPKPLIYAPSNLASGVPSRFSGSGSGTD | |
| FTLTISSLQPEDFATYYCQQWAFNPPTFGQGTKVE | |
| IKR | |

and 2H7.v511 variable heavy-domain sequence:

| | |
|---|---|
| EVQLVESGGGLVQPGGSLRLSCAASGYTFTSYNMHW | (SEQ ID NO:12) |
| VRQAPGKGLEWVGAIYPGNGATSYNQKFKGRFTISV | |
| DKSKNTLYLQMNSLRAEDTAVYYCARVVYYSYRYWY | |
| FDVWGQGTLVTVSS. | |

Where the humanized 2H7.v511 antibody is an intact antibody, it may comprise the light-chain amino acid sequence:

| | |
|---|---|
| DIQMTQSPSSLSASVGDRVTITCRASSSVSYLHWYQ | (SEQ ID NO:20) |
| QKPGKAPKPLIYAPSNLASGVPSRFSGSGSGTDFTL | |
| TISSLQPEDFATYYCQQWAFNPPTFGQGTKVEIKRT | |
| VAAPSVFIFPPSDEQLKSGTASVVCLLNNFYPREAK | |
| VQWKVDNALQSGNSQESVTEQDSKDSTYSLSSTLTL | |
| KADYEKHKVYACEVTHQGLSSPVTKSFNRGEC | |

and the heavy-chain amino acid sequence of SEQ ID NO:7 or:

| | |
|---|---|
| EVQLVESGGGLVQPGGSLRLSCAASGYTFTSYNMHW | (SEQ ID NO:13) |
| VRQAPGKGLEWVGAIYPGNGATSYNQKFKGRFTISV | |
| DKSKNTLYLQMNSLRAEDTAVYYCARVVYYSYRYWY | |
| FDVWGQGTLVTVSSASTKGPSVFPLAPSSKSTSGGT | |
| AALGCLVKDYFPEPVTVSWNSGALTSGVHTFPAVLQ | |
| SSGLYSLSSVVTVPSSSLGTQTYICNVNHKPSNTKV | |
| DKKVEPKSCDKTHTCPPCPAPELLGGPSVFLFPPKP | |
| KDTLMISRTPEVTCVVVDVSHEDPEVKFNWYVDGVE | |
| VHNAKTKPREEQYNATYRVVSVLTVLHQDWLNGKEY | |
| KCKVSNAALPAPIAATISKAKGQPREPQVYTLPPSR | |
| EEMTKNQVSLTCLVKGFYPSDIAVEWESNGQPENNY | |
| KTTPPVLDSDGSFFLYSKLTVDKSRWQQGNVFSCSV | |
| MHEALHNHYTQKSLSLSPG. | |

Unless indicated, the sequences disclosed herein of the humanized 2H7v.16 and variants thereof are of the mature polypeptide, i.e., without the leader sequence. (U.S. Patent Appl. Publication No. 2005/0095243).

BAFF Antagonists, BAFF Receptor Antagonists, and Other B-Cell Antagonists

BAFF (also known as BLyS, TALL-1, THANK, TNFSF13B, or zTNF4) is a member of the TNF1 ligand superfamily that is essential for B-cell survival and maturation. BAFF overexpression in transgenic mice leads to B-cell hyperplasia and development of severe autoimmune disease (Mackay, et al. (1999) J. Exp. Med. 190, 1697-1710; Gross, et al. (2000) Nature 404, 995-999; Khare, et al. (2000) Proc. Natl. Acad. Sci. U.S.A. 97, 3370-33752-4). BAFF acts on B-cells by binding to three members of the TNF receptor superfamily, TACI, BCMA, and BR3 (also known as BAFF-R) (Gross, et al., supra; Thompson, J. S., et al., (2001) Science 293, 2108-2111; Yan, M., et al., (2001) Curr. Biol. 11, 1547-1552; Yan, M., et al., (2000) Nat. Immunol. 1, 37-41; Schiemann, B., et al., (2001) Science 293, 2111-2114). Of the three BAFF receptors, only BR3 is specific for BAFF; the other two also bind the related TNF family member, APRIL. BR3 is a 184-residue type III transmembrane protein expressed on the surface of B-cells. (U.S. Patent Appl. Publication No. 2005/0095243).

According to certain embodiments of the invention, the B-cell antagonist is a BAFF antagonist or a BAFF receptor antagonist. The term “BAFF antagonist,” as used herein, includes any molecule that binds, associates, and/or interacts with a native sequence BAFF polypeptide and partially or fully blocks, inhibits, or neutralizes native sequence BAFF signaling. In selected embodiments, the present invention includes the use of antibodies or fragments thereof that bind to or associate with BAFF. Those skilled in the art will appreciate that native sequence BAFF polypeptide signaling promotes, among other things, B-cell survival and B-cell maturation. For example, a biologically active BAFF ligand potentiates any one or combination of the following events in vitro or in vivo: (i) an increased survival of B-cells; (ii) an increased level of IgG and/or IgM; (iii) an increased number of plasma cells; and (iv) processing of NF-κB2/100 to p52 NF-κB in splenic B-cells (e.g., Batten et al., J. Exp. Med. 192:1453-1465 (2000); Moore, et al., Science 285:260-263 (1999); Kayagaki et al., Immunity 17:515-524 (2002). Thus, the inhibition, blockage or neutralization of BAFF signaling results in, among other things, a reduction in the number of B-cells. Accordingly, a BAFF antagonist according to certain aspects of the invention will partially or fully block, inhibit, or neutralize one or more biological activities of a BAFF polypeptide in vitro or in vivo and thereby reduce or inhibit B-cell activity. Several assays useful for testing BAFF antagonists are described in U.S. Patent Appl. Publication No. 2005/0095243.

Peptides useful as antagonists of BAFF include, e.g., the peptide referred to as TALL-1 12-3 in WO 02/092620, having the following amino acid sequence:

| | |
|---|---|
| MLPGCKWDLLIKQWVCDPLGSGSATGGSGSTASSGS | (SEQ ID NO:14) |
| GSATHMLPGCKWDLLIKQWVCDPLGGGGGVDKTHTC | |
| PPCPAPELLGGPSVFLFPPKPKDTLMISRTPEVTCW | |
| WDVSHEDPEVKFNWYVDGVEVHNAKTKPREEQYNST | |
| YRWSVLTVLHQDWLNGKEYKCKVSNKALPAPIEKTI | |
| SKAKGQPREPQVYTLPPSRDELTKNQVSLTCLVKGF | |
| YPSDIAVEWESNGQPENNYKTTPPVLDSDGSFFLYS | |
| KLTVDKSRWQQGNVFSCSVMHEALHNHYTQKSLSLS | |
| PGK. | |

This peptide, as well as other exemplary peptides disclosed in WO 02/092620, binds BAFF and inhibits BAFF binding to its receptors, BR3, TACI and BCMA. The BAFF peptide antagonists set forth in WO 02/092620 may, in certain embodiments, be linked to, e.g., Fc or PEG.

Additional BAFF peptide antagonists include peptides or polypeptides comprising an amino acid sequence selected from the group consisting of: ECFDLLVRAWVPCSVLK (SEQ ID NO:15), ECFDLLVRHWVPCGLLR (SEQ ID NO:16), ECFDLLVRRWVPCEMLG (SEQ ID NO:17), ECFDLLVRSWVPCHMLR (SEQ ID NO:18), and ECFDLLVRHWVACGLLR (SEQ ID NO:19), and polypeptides comprising an amino acid sequence that is at least 40%, 45%, 50%, 55%, 60%, 65%, 70%, 75%, 80%, 85%, 90%, 91%, 92%, 93%, 94%, 95%, 96%, 97% 98% or 99% identical to any one of SEQ ID NOs: 15, 16, 17, 18 or 19.

Additional BAFF peptide antagonists that can be used in the practice of the methods of the present invention include polypeptides comprising an amino acid sequence of Formula I, Formula II or Formula III, as set forth in U.S. Patent Appl. Publication No. 2005/0095243.

In some embodiments of the invention, the BAFF antagonist is an anti-BAFF antibody, immunoadhesin or small molecule. The immunoadhesin, in certain embodiments comprises a BAFF binding region of a BAFF receptor (e.g., an extracellular domain of BR3, BCMA or TACI) in the form of a soluble construct. In a particularly preferred embodiment, the immunoadhesin is BR3-Fc, or polypeptides having a sequence of one of SEQ ID NOs: 15, 16, 17, 18 or 19 (as set forth in U.S. Patent Appl. Publication Nos. 2002/0037852, 2003/0059937, 2005/0095243 and 2005/0163775). In other embodiments, the immunoadhesin is a soluble form of TACI or BCMA (e.g., TACI-Fc, or BCMA-Fc).

Those skilled in the art will further appreciate that antibodies or fragments thereof that specifically bind to or associate with BAFF are also compatible with the teachings herein and are known in the art, e.g., in U.S. Patent Appl. Publication No. 2003/0059937. An exemplary antibody according to this aspect of the invention is LymphoStat-B™ (belimumab) (Human Genome Sciences, Inc.), a human monoclonal antibody that specifically recognizes and inhibits the biological activity of BAFF.

According to certain other embodiments of the invention, the B-cell antagonist is a BAFF receptor antagonist. The term “BAFF receptor antagonist,” as used herein, includes any molecule that binds or associates with a native sequence BAFF receptor (e.g., BR3, TACI or BCMA) polypeptide and/or partially or fully blocks, inhibits, or neutralizes native sequence BAFF signaling through the receptor. Thus, in selected embodiments of the invention, the B-cell antagonist comprises an antibody or fragment thereof, polypeptide or small molecule that binds or associates specifically with Btk, TACI, BCMA (U.S. Patent Appl. Publication No. 2002/0081296) or BAFF-R (U.S. Patent Appl. Publication No. 2002/0165156). Other exemplary B-cell antagonists include, e.g., antibodies, polypeptides or small molecules that inhibit the interaction of ITAM motifs from Ig-α/Ig-β with their targets, antibodies, polypeptides or small molecules that inhibit classical or alternative NFκB activation pathways, and antibodies, polypeptides or small molecules that inhibit OCA-B, CD40, LT-β, etc.

Conjugates and Other Modifications of B-Cell Antagonists

According to certain embodiments of the invention, the B-cell antagonist is conjugated to a cytotoxic agent.

Chemotherapeutic agents useful in the generation of B-cell antagonist-cytotoxic agent conjugates are well known in the art.

Conjugates of a B-cell antagonist and one or more small molecule toxins, such as a calicheamicin, a maytansine (U.S. Pat. No. 5,208,020), a trichothene, and CC 1065, are also contemplated herein. In one embodiment of the invention, the B-cell antagonist is conjugated to one or more maytansine molecules (e.g. about 1 to about 10 maytansine molecules per B-cell antagonist). Maytansine may, for example, be converted to May-SS-Me which may be reduced to May-SH3 and reacted with modified B-cell antagonists (Chari et al. Cancer Research 52: 127-131 (1992)) to generate a maytansinoid-B-cell antagonist conjugate.

Alternatively, the B-cell antagonist is conjugated to one or more calicheamicin molecules. The calicheamicin family of antibiotics are capable of producing double-stranded DNA breaks at sub-picomolar concentrations. Structural analogues of calicheamicin which may be used include, but are not limited to, γ₁^{I}, α₂^{I},α₃^{I}, N-acetyl-γ₁^{I} , PSAG and Φ₁^{I}(Hinman et al. Cancer Research 53: 3336-3342 (1993) and Lode et al. Cancer Research 58: 2925-2928 (1998)).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleuritesfordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993. Mytansinoids may also be conjugated to a B-cell antagonists.

The present invention further contemplates B-cell antagonists conjugated with a compound with nucleolytic activity (e.g. a ribonuclease or a DNA endonuclease such as a deoxyribonuclease; DNase).

A variety of radioactive isotopes are available for the production of radioconjugated B-cell antagonists. Examples include At²¹¹, I¹³¹, I¹²⁵, Y⁹⁰, Re¹⁸⁶, Re¹⁸⁸, Sm¹⁵³, Bi²¹², P³² and radioactive isotopes of Lu.

Conjugates of the B-cell antagonists and cytotoxic agents may be made using a variety of bifunctional protein coupling agents such as N-succinimidyl-3-(2-pyridyldithiol)propionate (SPDP), succinimidyl-4-(N-maleimidomethyl)cyclohexane-I-carboxylate, iminothiolane (IT), bifunctional derivatives of imidoesters (such as dimethyl adipimidate HCl), active esters (such as disuccinimidyl suberate), aldehydes (such as glutareldehyde), his-azido compounds (such as bis (p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al. Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the B-cell antagonist. See WO94/11026. The linker may be a “cleavable linker” facilitating release of the cytotoxic drug in the cell. For example, an acid-labile linker, peptidase-sensitive linker, dimethyl linker or disulfide-containing linker (Chari et al. Cancer Research 52:127-131 (1992)) may be used.

Alternatively, a fusion protein comprising the B-cell antagonist and cytotoxic agent may be made, e.g. by recombinant techniques or peptide synthesis.

In yet another embodiment, the B-cell antagonist may be conjugated to a “receptor” (such streptavidin) for utilization in “pretargeting” wherein the B-cell antagonist-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

The B-cell antagonists of the present invention may also be conjugated with a prodrug-activating enzyme which converts a prodrug (e.g. a peptidyl chemotherapeutic agent, see WO81/01145) to an active drug. See, for example, WO 88/07378 and U.S. Pat. No. 4,975,278. The enzyme component of such conjugates includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine dearninase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-ammo acid substituents; carbohydrate-cleaving enzymes such as O-galactosidase and neuraminidase useful for converting glycosylated prodrugs into free drugs; P-lactamase useful for converting drugs derivatized with P-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328: 457-458 (1987)). B-cell antagonist-abzyme conjugates can be prepared as described herein for delivery of the abzyme to a cell population or tissue.

Enzymes can be covalently bound to the B-cell antagonist by techniques well known in the art such as the use of the heterobifunctional crosslinking reagents. Alternatively, fusion proteins comprising at least the antigen binding region of a B-cell antagonist of the invention linked to at least a functionally active portion of an enzyme can be constructed using recombinant DNA techniques well known in the art (see, e.g., Neuberger et al., Nature 312: 604-608 (1984)).

Other modifications of the B-cell antagonists are contemplated herein. For example, the B-cell antagonist may be linked to one of a variety of nonproteinaceous polymers, e.g., polyethylene glycol, polypropylene glycol, polyoxyalkylenes, or copolymers of polyethylene glycol and polypropylene glycol.

An exemplary polymer that can be used for conjugation to a B-cell antagonist is a polyalkylene glycol (PEG). PEG moieties, e.g., 1, 2, 3, 4 or 5 PEG polymers, can be conjugated to each B-cell antagonist to increase serum half life, as compared to the B-cell antagonist alone. PEG moieties are non-antigenic and essentially biologically inert. PEG moieties used in the practice of the invention may be branched or unbranched.

The number of PEG moieties attached to the B-cell antagonist and the molecular weight of the individual PEG chains can vary. In general, the higher the molecular weight of the polymer, the fewer polymer chains attached to the polypeptide. Usually, the total polymer mass attached to the B-cell antagonist is from 20 kDa to 40 kDa. Thus, if one polymer chain is attached, the molecular weight of the chain is generally 20-40 kDa. If two chains are attached, the molecular weight of each chain is generally 10-20 kDa. If three chains are attached, the molecular weight is generally 7-14 kDa.

The polymer, e.g., PEG, can be linked to the B-cell antagonist through any suitable, exposed reactive group on the polypeptide. The exposed reactive group(s) can be, e.g., an N-terminal amino group or the epsilon amino group of an internal lysine residue, or both. An activated polymer can react and covalently link at any free amino group on the B-cell antagonist. Free carboxylic groups, suitably activated carbonyl groups, hydroxyl, guanidyl, imidazole, oxidized carbohydrate moieties and mercapto groups of the B-cell antagonist (if available) also can be used as reactive groups for polymer attachment.

In a conjugation reaction, from about 1.0 to about 10 moles of activated polymer per mole of polypeptide, depending on polypeptide concentration, is typically employed. Usually, the ratio chosen represents a balance between maximizing the reaction while minimizing side reactions (often non-specific) that can impair the desired pharmacological activity of the B-cell antagonist. Preferably, at least 50% of the biological activity (as demonstrated, e.g., in any of the assays described herein or known in the art) of the B-cell antagonist is retained, and most preferably nearly 100% is retained.

The polymer can be conjugated to the B-cell antagonist using conventional chemistry. For example, a polyalkylene glycol moiety can be coupled to a lysine epsilon amino group of the B-cell antagonist. Linkage to the lysine side chain can be performed with an N-hydroxylsuccinimide (NHS) active ester such as PEG succinimidyl succinate (SS-PEG) and succinimidyl propionate (SPA-PEG). Suitable polyalkylene glycol moieties include, e.g., carboxymethyl-NHS and norleucine-NHS, SC. These reagents are commercially available. Additional amine-reactive PEG linkers can be substituted for the succinimidyl moiety. These include, e.g., isothiocyanates, nitrophenylcarbonates (PNP), epoxides, benzotriazole carbonates, SC-PEG, tresylate, aldehyde, epoxide, carbonylimidazole and PNP carbonate. Conditions are usually optimized to maximize the selectivity and extent of reaction. Such optimization of reaction conditions is within ordinary skill in the art.

PEGylation can be carried out by any of the PEGylation reactions known in the art. See, e.g., Focus on Growth Factors, 3: 4-10, 1992 and European patent applications EP 0 154 316 and EP 0 401 384. PEGylation may be carried out using an acylation reaction or an alkylation reaction with a reactive polyethylene glycol molecule (or an analogous reactive water-soluble polymer).

PEGylation by acylation generally involves reacting an active ester derivative of polyethylene glycol. Any reactive PEG molecule can be employed in the PEGylation. PEG esterified to N-hydroxysuccinimide (NHS) is a frequently used activated PEG ester. As used herein, “acylation” includes without limitation the following types of linkages between the therapeutic protein and a water-soluble polymer such as PEG: amide, carbamate, urethane, and the like. See, e.g., Bioconjugate Chem. 5: 133-140, 1994. Reaction parameters are generally selected to avoid temperature, solvent, and pH conditions that would damage or inactivate the B-cell antagonist.

Generally, the connecting linkage is an amide and typically at least 95% of the resulting product is mono-, di- or tri-PEGylated. However, some species with higher degrees of PEGylation may be formed in amounts depending on the specific reaction conditions used. Optionally, purified PEGylated species are separated from the mixture, particularly unreacted species, by conventional purification methods, including, e.g., dialysis, salting-out, ultrafiltration, ion-exchange chromatography, gel filtration chromatography, hydrophobic exchange chromatography, and electrophoresis.

PEGylation by alkylation generally involves reacting a terminal aldehyde derivative of PEG with an B-cell antagonist of the invention in the presence of a reducing agent. In addition, one can manipulate the reaction conditions to favor PEGylation substantially only at the N-terminal amino group of the B-cell antagonist, i.e. a mono-PEGylated protein. In either case of mono-PEGylation or poly-PEGylation, the PEG groups are typically attached to the protein via a —CH₂—NH— group. With particular reference to the —CH₂— group, this type of linkage is known as an “alkyl” linkage.

Derivatization via reductive alkylation to produce an N-terminally targeted mono-PEGylated product exploits differential reactivity of different types of primary amino groups (lysine versus the N-terminal) available for derivatization. The reaction is performed at a pH that allows one to take advantage of the pKa differences between the epsilon-amino groups of the lysine residues and that of the N-terminal amino group of the protein. By such selective derivatization, attachment of a water-soluble polymer that contains a reactive group, such as an aldehyde, to a protein is controlled: the conjugation with the polymer takes place predominantly at the N-terminus of the protein and no significant modification of other reactive groups, such as the lysine side chain amino groups, occurs.

The polymer molecules used in both the acylation and alkylation approaches are selected from among water-soluble polymers. The polymer selected is typically modified to have a single reactive group, such as an active ester for acylation or an aldehyde for alkylation, so that the degree of polymerization may be controlled as provided for in the present methods. An exemplary reactive PEG aldehyde is polyethylene glycol propionaldehyde, which is water stable, or mono C₁-C₁₀ alkoxy or aryloxy derivatives thereof (see, e.g., Harris et al., U.S. Pat. No. 5,252,714). The polymer may be branched or unbranched. For the acylation reactions, the polymer(s) selected typically have a single reactive ester group. For reductive alkylation, the polymer(s) selected typically have a single reactive aldehyde group. Generally, the water-soluble polymer will not be selected from naturally occurring glycosyl residues, because these are usually made more conveniently by mammalian recombinant expression systems.

Methods for preparing a PEGylated B-cell antagonist s of the invention generally includes the steps of (a) reacting an B-cell antagonist of the invention with polyethylene glycol (such as a reactive ester or aldehyde derivative of PEG) under conditions whereby the molecule becomes attached to one or more PEG groups, and (b) obtaining the reaction product(s). In general, the optimal reaction conditions for the acylation reactions will be determined case-by-case based on known parameters and the desired result. For example, a larger the ratio of PEG to protein, generally leads to a greater the percentage of poly-PEGylated product.

Reductive alkylation to produce a substantially homogeneous population of mono-polymer/B-cell antagonist generally includes the steps of: (a) reacting an B-cell antagonist of the invention with a reactive PEG molecule under reductive alkylation conditions, at a pH suitable to pen-nit selective modification of the N-terminal amino group of NgR; and (b) obtaining the reaction product(s).

For a substantially homogeneous population of mono-polymer/B-cell antagonist, the reductive alkylation reaction conditions are those that permit the selective attachment of the water-soluble polymer moiety to the N-terminus of an B-cell antagonist of the invention. Such reaction conditions generally provide for pKa differences between the lysine side chain amino groups and the N-terminal amino group. For purposes of the present invention, the pH is generally in the range of 3-9, typically 3-6.

B-cell antagonists of the invention can include a tag, e.g., a moiety that can be subsequently released by proteolysis. Thus, the lysine moiety can be selectively modified by first reacting a His-tag modified with a low-molecular-weight linker such as Traut's reagent (Pierce Chemical Company, Rockford, Ill.) which will react with both the lysine and N-terminus, and then releasing the His tag. The polypeptide will then contain a free SH group that can be selectively modified with a PEG containing a thiol-reactive head group such as a maleimide group, a vinylsulfone group, a haloacetate group, or a free or protected SH.

Traut's reagent can be replaced with any linker that will set up a specific site for PEG attachment. For example, Traut's reagent can be replaced with SPDP, SMPT, SATA, or SATP (Pierce Chemical Company, Rockford, Ill.). Similarly one could react the protein with an amine-reactive linker that inserts a maleimide (for example SMCC, AMAS, BMPS, MBS, EMCS, SMPB, SMPH, KMUS, or GMBS), a haloacetate group (SBAP, SIA, SIAB), or a vinylsulfone group and react the resulting product with a PEG that contains a free SH.

In some embodiments, the polyalkylene glycol moiety is coupled to a cysteine group of the B-cell antagonist. Coupling can be effected using, e.g., a maleimide group, a vinylsulfone group, a haloacetate group, or a thiol group.

Optionally, the B-cell antagonist is conjugated to the polyethylene-glycol moiety through a labile bond. The labile bond can be cleaved in, e.g., biochemical hydrolysis, proteolysis, or sulfhydryl cleavage. For example, the bond can be cleaved under in vivo (physiological) conditions.

The reactions may take place by any suitable method used for reacting biologically active materials with inert polymers, generally at about pH 5-8, e.g., pH 5, 6, 7, or 8, if the reactive groups are on the alpha amino group at the N-terminus. Generally the process involves preparing an activated polymer and thereafter reacting the protein with the activated polymer to produce the soluble protein suitable for formulation.

The B-cell antagonists disclosed herein may also be formulated as liposomes. Liposomes containing the B-cell antagonist are prepared by methods known in the art, such as described in Epstein et al., Proc. Natl. Acad. Sci. USA, 82:3688 (1985); Hwang et al., Proc. Natl Acad. Sci. USA 77:4030 (1980); U.S. Pat. Nos. 4,485,045 and 4,544,545; and WO97/38731. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatizedphosphatidylethanolamine (PEG-PE). Liposomes are extruded through filters of defined pore size to yield liposomes with the desired diameter. Fab′ fragments of an antibody of the present invention can be conjugated to the liposomes as described in Martin et al. J. Biol. Chem. 257: 286-288 (1982) via a disulfide interchange reaction. A chemotherapeutic agent is optionally contained within the liposome. See Gabizon et al. J. National Cancer Inst. 81(19):1484 (1989).

Amino acid sequence modification(s) of protein or peptide B-cell antagonists described herein are contemplated. For example, it may be desirable to improve the binding affinity and/or other biological properties of the B-cell antagonist.

Amino acid sequence variants of the B-cell antagonist are prepared by introducing appropriate nucleotide changes into the B-cell antagonist-encoding nucleic acid, or by peptide synthesis. Such modifications include, for example, deletions from, and/or insertions into and/or substitutions of, residues within the amino acid sequences of the B-cell antagonist. Any combination of deletion, insertion, and substitution is made to arrive at the final construct, provided that the final construct possesses the desired characteristics. The amino acid changes also may alter post-translational processes of the B-cell antagonist, such as changing the number or position of glycosylation sites.

A useful method for identification of certain residues or regions of the B-cell antagonist that are preferred locations for mutagenesis is called “alanine scanning mutagenesis” as described by Cunningham and Wells Science 244:1081-1085 (1989). Here, a residue or group of target residues are identified (e.g., charged residues such as arg, asp, his, lys, and glu) and replaced by a neutral or negatively charged amino acid (most preferably alanine or polyalanine). Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed B-cell antagonist variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include a B-cell antagonist with an N-terminal methionyl residue or the B-cell antagonist fused to a cytotoxic polypeptide. Other insertional variants of the B-cell antagonist include the fusion to the N— or C-terminus of the B-cell antagonist of an enzyme, or a polypeptide which increases the serum half-life of the B-cell antagonist.

Another type of variant is an amino acid substitution variant. These variants have at least one amino acid residue in the B-cell antagonist molecule replaced by different residue. The sites of greatest interest for substitutional mutagenesis of antibody B-cell antagonists include the hypervariable regions, but FR alterations are also contemplated.

Conservative substitutions are shown in Table 2 under the heading of “preferred substitutions.” If such substitutions result in a change in biological activity, then more substantial changes, denominated “exemplary substitutions” in Table 2, or as further described below in reference to amino acid classes, may be introduced and the products screened.

TABLE 2

| Original | | Preferred |
|---|---|---|
| Residue | Exemplary Substitutions | Substitutions |
| Ala (A) | val; leu; ile | val |
| Arg (R) | lys; gln; asn | lys |
| Asn (N) | gln; his; asp; lys; arg | gln |
| Asp (D) | glu; asn; | glu |
| Cys (C) | ser; ala | ser |
| Gln (Q) | asn; glu | asn |
| Glu (E) | asp; gln | asp |
| Gly (G) | ala | ala |
| His (H) | asn, gln, lys, arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Tpr (W) | tyr; phe | tyr |
| Tyr (Y) | trp; phe; thr; ser | phe |
| Val (V) | ile; leu; met; phe; ala; norleucine | leu |

Substantial modifications in the biological properties of the B-cell antagonists are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;

(2) neutral hydrophilic: cys, ser, thr;

(3) acidic: asp, glu;

(4) basic: asn, gin, his, lys, arg;

(5) residues that influence chain orientation: gly, pro; and

(6) aromatic: trp, tyr, phe.

Non-conservative substitutions will entail exchanging a member of one of these classes for another class.

Any cysteine residue not involved in maintaining the proper conformation of the B-cell antagonist also may be substituted, generally with serine, to improve the oxidative stability of the molecule and prevent aberrant crosslinking. Conversely, cysteine bond(s) may be added to the B-cell antagonist to improve its stability (particularly where the B-cell antagonist is an antibody fragment such as an Fv fragment).

A particularly preferred type of substitutional variant involves substituting one or more hypervariable region residues of a parent antibody. Generally, the resulting variant(s) selected for further development will have improved biological properties relative to the parent antibody from which they are generated. A convenient way for generating such substitutional variants is affinity maturation using phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity) as herein disclosed. In order to identify candidate hypervariable region sites for modification, alanine scanning mutagenesis can be performed to identify hypervariable region residues contributing significantly to antigen binding. Alternatively, or in additionally, it may be beneficial to analyze a crystal structure of the antigen-antibody complex to identify contact points between the antibody and antigen. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Another type of amino acid variant of the B-cell antagonist alters the original glycosylation pattern of the B-cell antagonist. Such altering includes deleting one or more carbohydrate moieties found in the B-cell antagonist, and/or adding one or more glycosylation sites that are not present in the B-cell antagonist.

Glycosylation of polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the B-cell antagonist is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original B-cell antagonist (for O-linked glycosylation sites).

Where the antibody comprises an Fc region, the carbohydrate attached thereto may be altered. For example, antibodies with a mature carbohydrate structure which lacks fucose attached to an Fc region of the antibody are described in US Pat Appl. Publication No. U.S. 2003/0157108. Antibodies with a bisecting N-acetylglucosamine (GlcNAc) in the carbohydrate attached to an Fc region of the antibody are referenced in WO03/011878, and U.S. Pat. No. 6,602,684. Antibodies with at least one galactose residue in the oligosaccharide attached to an Fc region of the antibody are reported in WO97/30087. See, also, WO98/58964 and WO99/22764 concerning antibodies with altered carbohydrate attached to the Fc region thereof.

Nucleic acid molecules encoding amino acid sequence variants of the B-cell antagonist are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the B-cell antagonist.

It may be desirable to modify the B-cell antagonist of the invention with respect to effector function, e.g. so as to enhance antigen-dependent cell-mediated cyotoxicity (ADCC) and/or complement dependent cytotoxicity (CDC) of the B-cell antagonist. This may be achieved by introducing one or more amino acid substitutions in an Fc region of an antibody B-cell antagonist. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-fibrotic activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al. Anti-Cancer Drug Design 3:219-230 (1989). WO00/42072 describes antibodies with improved ADCC function in the presence of human effector cells, where the antibodies comprise amino acid substitutions in the Fc region thereof.

Antibodies with altered Clq binding and/or complement dependent cytotoxicity (CDC) are described in WO99/51642, U.S. Pat. No. 6,194,551B1, U.S. Pat. No. 6,242,195B1, U.S. Pat. No. 6,528,624B1 and U.S. Pat. No. 6,538,124. The antibodies comprise an amino acid substitution at one or more of amino acid positions 270, 322, 326, 327, 329, 313, 333 and/or 334 of the Fc region thereof.

To increase the serum half life of the B-cell antagonist, one may incorporate a salvage receptor binding epitope into the B-cell antagonist (especially an antibody fragment) as described in U.S. Pat. No. 5,739,277, for example. As used herein, the term “salvage receptor binding epitope” refers to an epitope of the Fc region of an IgG molecule (e.g., IgG.sub.1, IgG.sub.2, IgG.sub.3, or IgG.sub.4) that is responsible for increasing the in vivo serum half-life of the IgG molecule. Antibodies with substitutions in an Fc region thereof and increased serum half-lives are also described in WO00/42072.

Engineered antibodies with three or more (preferably four) functional antigen binding sites are also contemplated (U.S. Patent Appl. Publication No. U.S. 2002/0004587).

Formulation and Administration of B-Cell Antagonists

B-cell antagonists of the invention are preferably administered to patients in the form of therapeutic formulations. Therapeutic formulations of the B-cell antagonists used in accordance with the present invention are prepared for storage by mixing a B-cell antagonist having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Exemplary anti-CD20 antibody formulations are described in WO98/56418. This publication describes a liquid multidose formulation comprising 40 mg/mL rituximab, 25 mM acetate, 150 mM trehalose, 0.9% benzyl alcohol, 0.02% polysorbate 20 at pH 5.0 that has a minimum shelf life of two years storage at 2-8° C. Another anti-CD20 formulation comprises 10 mg/mL rituximab in 9.0 mg/mL sodium chloride, 7.35 mg/mL sodium citrate dihydrate, 0.7 mg/mL polysorbate 80, and Sterile Water for Injection, pH 6.5.

Lyophilized formulations adapted for subcutaneous administration are described in U.S. Pat. No. 6,267,958. Such lyophilized formulations may be reconstituted with a suitable diluent to a high protein concentration and the reconstituted formulation may be administered subcutaneously to the patient to be treated herein.

The formulation herein may also contain more than one active compound as necessary for the particular indication being treated, preferably those with complementary activities that do not adversely affect each other. For example, it may be desirable to further provide a cytotoxic agent, chemotherapeutic agent, cytokine, inhibitor of the TGF-β pathway (e.g., monoclonal antibody, peptide, small molecule antagonist, inhibitor of TGF-β activation), integrin receptor antagonist, or immunosuppressive agent (e.g., one which acts on T cells, such as cyclosporin or an antibody that binds T cells, e.g. one which binds LFA-1). The effective amount of such other agents depends on the amount of B-cell antagonist present in the formulation, the type of disease or disorder or treatment, and other factors.

The B-cell antagonists may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of B-cell antagonists may be prepared. Suitable examples of sustained-release preparations include semipermeable matrices of solid hydrophobic polymers containing the B-cell antagonist, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The B-cell antagonist may be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the B-cell antagonist may suitably be administered by pulse infusion, e.g., with declining doses of the B-cell antagonist. Preferably the dosing is given by injections, most preferably intravenous or subcutaneous injections, depending in part on whether the administration is brief or chronic.

In certain exemplary embodiments of the invention, the B-cell antagonists are administered to the patient (e.g., intravenously) in a dosage of between 1 mg/m² and 500 mg/m². For instance, the B-cell antagonist may be administered in a dosage of 1 mg/m², 2 mg/m², 3 mg/m², 4 mg/m², 5 mg/m², 10 mg/m², 15 mg/m², 20 mg/m², 25 mg/m², 30 mg/m², 35 mg/m², 40 mg/m², 45 mg/m², 50 mg/m², 55 mg/m², 60 mg/m², 65 mg/m², 70 mg/m², 75 mg/m², 80 mg/m², 85 mg/m², 90 mg/m², 95 mg/m², 100 mg/m², 105 mg/m², 110 mg/m², 115 mg/m², 120 mg/m², 125 mg/m², 130 mg/m², 135 mg/m², 140 mg/m², 145 mg/m², 150 mg/m², 155 mg/m², 160 mg/m², 165 mg/m², 170 mg/m², 175 mg/m², 180 mg/m², 185 mg/m², 190 mg/m², 195 mg/m², 200 mg/m², 205 mg/m², 210 mg/m², 215 , mg/m², 220 mg/m², 225 mg/m², 230 mg/m², 235 mg/m², 240 mg/m², 245 mg/m², 250 mg/m², 255 mg/m², 260 mg/m², 265 mg/m², 270 mg/m², 275 mg/m², 280 mg/m², 285 mg/m², 290 mg/m², 295 mg/m², 300 mg/m², 305 mg/m², 310 mg/m², 315 mg/m², 320 mg/m², 325 mg/m², 330 mg/m², 335 mg/m², 340 mg/m², 345 mg/m², 350 mg/m², 355 mg/m², 360 mg/m², 365 mg/m², 370 mg/m², 375 mg/m², 380 mg/m², 385 mg/m², 390 mg/m², 395 mg/m² or 400 mg/m².

The B-cell antagonist can be administered according to a wide variety of dosing schedules. (See, e.g., U.S. Patent Appl. Publication No. 2006/0002930). For example, the B-cell antagonist can be administered once daily for a predetermined amount of time (e.g., four to eight weeks, or more), or according to a weekly schedule (e.g., one day per week, two days per week, three days per week, four days per week, five days per week, six days per week or seven days per week) for a predetermined amount of time (e.g., four to eight weeks, or more). A specific example of a “once weekly” dosing schedule is administration of the B-cell antagonist on days 1, 8, 15 and 22 of the treatment period. In alternative embodiments the B-cell antagonist may be administered intermittently over a period of months. For example, the B-cell antagonist may be administered weekly for three consecutive weeks biannually (i.e. repeat the weekly dosing schedule every six months). It will be appreciated that such administration regimens may be continued for extended periods (on the order of years) to maintain beneficial therapeutic effects provided by initial treatments. In yet other embodiments such maintenance therapy may be effected following an acute dosing regimen designed to reduce the immediate symptoms of the fibrotic condition.

The amount of B-cell antagonist administered each time throughout the treatment period can be the same; alternatively, the amount administered each time during the treatment period can vary (e.g., the amount administered at a given time can be more or less than the amount administered previously). For example, doses given during maintenance therapy may be lower than those administered during the acute phase of treatment. Appropriate dosing schedules depending on the specific circumstances will be apparent to persons of ordinary skill in the art.

Aside from administration of protein B-cell antagonists to the patient the present application contemplates administration of B-cell antagonists by gene therapy. Such administration of nucleic acid encoding the B-cell antagonist is encompassed by the expression “administering to a patient in need of such treatment a therapeutically effective amount of a B-cell antagonist.” See, for example, WO96/07321, concerning the use of gene therapy to generate intracellular antibodies.

There are two major approaches to getting the nucleic acid (optionally contained in a vector) into the patient's cells; in vivo and ex vivo. For in vivo delivery the nucleic acid is injected directly into the patient, usually at the site where the B-cell antagonist is required. For ex vivo treatment, the patient's cells are removed, the nucleic acid is introduced into these isolated cells and the modified cells are administered to the patient either directly or, for example, encapsulated within porous membranes which are implanted into the patient (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retrovirus.

Exemplary in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. USA 87:3410-3414 (1990). For review of gene marking and gene therapy protocols see Anderson et al., Science 256:808-cell 813 (1992). See also WO 93/25673 and the references cited therein.

Combinations of B-Cell Antagonists and Other Agents

In certain embodiments of the invention, multiple types of B-cell antagonists are combined with one another and administered to a patient to treat one or more fibrosis conditions. For example, the invention includes methods for treating fibrosis conditions that comprise administering to a patient a therapeutically effective amount of an antibody against CD20 (e.g., rituximab) and a BAFF antagonist as described elsewhere herein and in U.S. Patent Appl. Publication No. 2005/0095243 which is incorporated by reference herein in its entirety. When multiple B-cell antagonists are administered to a patient, the different B-cell antagonists can be administered together in a single pharmaceutical composition, or, more preferably, can be administered sequentially in separate dosages and in any order.

The present invention also includes methods for treating fibrosis conditions that comprise administering to a patient in need thereof a combination comprising a first agent and a second agent, wherein the first agent is a B-cell antagonist and the second agent is an agent that is useful for treating one or more fibrosis conditions but is not necessarily a B-cell antagonist. For example, according to certain embodiments of the invention, a B-cell antagonist is administered to a patient along with an antagonist of one or more integrin receptors (e.g., α₁β₁, αᵥβ₆, αᵥβ₈, αᵥβ₅, α_{5β}₁, α₄β₁, α₄β₇, etc.), including antibodies, polypeptide antagonists and/or small molecule antagonists specific for one or more integrin receptors (e.g., α₁β₁, αᵥβ₆, αᵥβ₈, αᵥβ₅, α_{5β}₁, α₄β₁, α₄β₇, etc.). (U.S. Pat. Nos. 6,652,856 and 6,692,741, and U.S. Patent Appl. Publication Nos. 2004/0248837, 2004/0208878, 2002/0004482, 2005/0255102, and 2005/0226885). An exemplary antibody that specifically binds to the α₄β₁ integrin receptor and that can be used in combination with a B-cell antagonist for the treatment of a fibrotic condition in the context of the present invention is natalizumab (Tysabri®) as set forth in U.S. published application No. 2005/0276803.

In certain embodiments of this aspect of the invention, the second agent that is administered with a B-cell antagonist is, e.g., a steroid, a cytotoxic agent, colchicine, oxygen, an antioxidant (e.g., N-acetylcysteine), a metal chelator (e.g., terathiomolybdate), IFN-γ, or alpha-antitrypsin. The second agent, in certain embodiments, may be an inhibitor of Btk, including, e.g., small molecule inhibitors of Btk. The second agent, in certain embodiments, may be an inhibitor of TWEAK, including, e.g., antibodies and small molecule inhibitors of TWEAK. In still other embodiments, the second agent may comprise an LTBR antagonist (e.g., a soluble fusion protein or antibody); see U.S. Pat. Nos. 7,030,080 and 7,001,921; or an antagonist of TRAIL-R2.

According to certain embodiments of this aspect of the invention, the second agent that is administered with a B-cell antagonist may be, e.g., a TGF-β pathway inhibitor. Exemplary TGF-β pathway inhibitors that can be used in the context of the present invention include, but are not limited to, antibodies, synthetic or native sequence peptides and small molecules that inhibit or antagonize one or more components of the TGF-β signaling pathway including, e.g., Ang II, IL-1, IL-4, IL-10, IL-13, MIF, PDGF, RAGE, AGE, TNF-α, Thrombospondin-1, VLA-1, SMAD-2, SMAD-3 (U.S. Patent Appl. Publication No. 2003/0139366), SMAD-4, ERK, p15, Ink4b, p21 Waf1, p27Kip1, p-38, CTGF (U.S. Patent Appl. Publication No. 2004/0248206), PAI-1, PTHrP, Endothelin-1, Farnesoid X, HGF, IGF-1, MMP-1, MMP-9, PGE2, Propyl Hydroxylase, Procollagens, Fibrillin, TIMP, CXCR4, CXCL12, CCR2, CCL2, CCL-7 and CCL-22. Other exemplary TGF-β pathway inhibitors that can be used in the context of the present invention include, e.g., TGF-β ligand and receptor antagonists, including, e.g., antibodies, soluble TGF-β RII-Fc fusion proteins, LAP-Fc fusion proteins, TGF-β RI or RII kinase inhibitors, and small molecule inhibitors downstream of TGF-β RII.

Additional agents that may be administered with a B-cell antagonist in the context of the present invention include, e.g., pirfenidone, endothelin antagonists, TNF-α inhibitors, PDGF inhibitors, CTGF inhibitors, CD40 ligand antagonists (U.S. Pat. No. 6,506,383), BCMA-Ig, P38 MAP kinase inhibitors, prednisone, cytoxan, and azathioprine.

Specific exemplary clinical products that can be used in combination with a B-cell antagonist to treat fibrosis conditions in the context of the present invention include those listed in Table 3.

TABLE 3

| Product Name | Description | Developed By |
|---|---|---|
| trastuzumab (Herceptin ®) | anti-Her2/neu antibody | Genentech |
| pertuzumab (Omnitarg ™) | anti-Her2 antibody | Genentech |
| cetuximab (Erbitux ®) | chimeric anti-EGFR antibody | Imclone |
| gemtuzumab ozogamicin | anti-CD33 (p67) antibody | Celltech/Wyeth |
| (Mylotarg ®) | | |
| alefacept (Amevive ®) | anti-LFA-3 Fc fusion | Biogen Idec |
| infliximab (Remicade ®) | anti-TNF-α antibody | Centocor |
| adalimumab (Humira ®) | anti-TNF-α antibody | Abbott |
| etanercept (Enbrel ®) | anti-TNF-α Fc fusion | Immunex/Amgen |
| natalizumab (Tysabri ®) | anti-α4-β1 (VLA-4) and α4-β7 | Biogen Idec |
| | antibody | |
| bevacizumab (Avastin ™) | anti-VEGF antibody | Genentech |
| omalizumab (Xolair ™) | anti-IgE antibody | Genentech |
| efalizumab (Raptiva ™) | anti-CD11 antibody | Genentech/Xoma |
| labetuzumab (CEA-Cide ™) | anti-carcinoembryonic antigen | Immunomedics |
| | (CEA) antibody | |
| epratuzumab | anti-CD22 antibody | Immunomedics |
| (LymphoCide ™) | | |
| visilizumab (Nuvion ®) | anti-CD3 antibody | PDL |
| HuZAF ™ | anti-gamma interferon antibody | PDL |
| imatinib mesylate | Bcr-Ab1 tyrosine kinase inhibitor | Novartis |
| (Gleevec ™) | | |
| bosentan (Tracleer ®) | endothelin inhibitor | Actelion |
| interferon gamma-1b | immune system stimulator | Intermune |
| (Actimmune ®) | | |
| abatacept (Orencia ®) | CTLA-4-Fc fusion protein | Bristol-Myers |
| | | Squibb |

Kits

The present invention also includes kits for treating fibrosis conditions. The kits of the invention comprise one or more containers wherein at least one of the containers comprises a B-cell antagonist. Any of the B-cell antagonists described elsewhere herein may be included within the kits of the invention. The kits of the invention may also comprise one or more containers comprising one or more additional agents that can be administered in combination with a B-cell antagonist to treat a fibrosis condition. Such additional agents are described elsewhere herein. The kits may optionally comprise one or more sets of instructions for treating a fibrosis condition. The instructions may include, inter alia, information pertaining to the amount of B-cell antagonist and/or other agents to be administered to a patient, the timing and frequency of administration, the suggested routes of administration, and the characteristics and/or symptoms displayed by patients to whom the B-cell antagonist and/or other agents should be administered.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Attenuated Liver Fibrosis in the Absence of B-Cells

Introduction

The hallmarks of chronic liver diseases, such as alcohol-induced liver degeneration, hepatitis C infection, non-alcohol induced steatohepatitis, are chronic inflammation, cellular damage, regeneration and fibrosis. All of these features can be evoked by repeated carbon tetrachloride (CCl₄) induced liver injury. (Jungermann and Katz, Physiol. Rev. 69:708-764 (1989); Friedman, Semin. Liver Dis. 19:129-140 (1999)). In this Example, CCl₄-induced fibrosis was assessed in wild-type and B-cell deficient mice.

In an alternative model, liver injury is induced by a biliary toxin α-naphthylisothiocyanate (ANIT), mimicking biliary cirrhosis and sclerosing cholangitis. (Tjandra et al., Hepatology 31:280-290 (2000)). ANIT, similar to CCl₄, induces non-immune cell targeted hepatotoxicity followed by inflammatory and fibrotic responses, however at a different hepatic anatomic location compared to CCl₄.

Following 6 weeks of CCl₄ treatment, histochemical analyses showed markedly reduced collagen deposition in the B-cell deficient mice compared to similarly treated wild-type mice. In addition, by analyzing mice that have normal numbers of B-cells but lack T-cells, it was established that B-cells contribute to fibrosis in a T-cell-independent manner. The ANIT treated JH −/− mice showed similar results with respect to collagen deposition.

Materials And Methods

Mice

Unless otherwise stated, mice were kept in a specific pathogen free mouse facility at Biogen Idec (Cambridge, Mass.). All animal procedures were approved by Biogen Idec's Institutional Animal Care and Use Committee. Male mice of the strains listed in Table 4 had to weigh 20 g or more and be at least 6 wks of age to be included in the study.

TABLE 4

| Mutant | Control | Commercial Source |
|---|---|---|
| J_{H}-/-(001147-M) | BALB/c (BALB) | Taconic, Germantown, |
| | | NY |
| MHCII-/-(ABBN12- | C57BL/6Tac (B6) | Taconic |
| M) | | |
| b2m-/-(B2MN12-M) | C57BL/6Tac (B6) | Taconic |
| RAG2-/-(000601-M) | BALB/c (BALB) | Taconic |
| TCRδ-/-(002120) | C57BL/6 (000664) | The Jackson |
| | | Laboratory, Bar Harbor, |
| | | ME |
| BAFFtg* | C57BL/6 (000664) | The Jackson Laboratory |
| mIgM-Tg^{¶} | BALB/c (BALB) | Taconic |
| LPM2a^{§} | BALB/cAnNCrlBr | Charles River, |
| | | Wilmington, MA |

*See Mackay et al., J. Exp. Med. 190: 1697-1710 (1999).
^{¶}See Chan et al., J. Exp. Med. 189: 1639-1648 (1999).
^{§}See Casola et al., Nat. Immunol. 5: 317-327 (2004).

CCl₄ and ANIT Injury Models

A mix of CCl₄ (Sigma-Aldrich Corp., St. Louis, Mo.) with mineral oil (Sigma-Aldrich Corp.) was delivered by gavage in a volume not exceeding 0.2 ml with a 20 gauge animal feeding needle. Experiments were performed using a 3.5 mg/kg or 1.75 mg/kg dose of CCl₄. The latter dose was preferred because it reduced morbidity/mortality and still induced changes in serum alanine aminotransferase (ALT) levels and collagen deposition comparable to the higher dose. For a long-term experiment, mice were gavaged once a week for 6 weeks. Short-term experiments included one CCl₄ administration.

ANIT (1-naphthyl isothiocyanate, Sigma-Aldrich Corp.) was dissolved in mineral oil (Sigma-Aldrich Corp.) at 30 mg/ml. Mice were gavaged with 50 mg/kg twice a week, for 8 weeks.

Serum ALT levels were measured 24 hrs after CCl₄ administration. One week after the 6^{th} weekly gavage or on the indicated day after a single gavage, mice were sacrificed and three different liver lobes were taken and from each mouse and incubated in 4% PFA in PBS for 2 days prior to embedding for further immunohistochemical analysis.

Liver Lymphocyte Isolation

Mice were euthanized by CO₂ inhalation. The hepatic portal vein was cannulated with a 25 G needle and perfused with 10 ml of cold PBS. After removal of the gall bladder, the liver was cut into segments and passed through a 70 μm mesh cell strainer (BD Falcon, Bedford, Mass.) in 50 ml of ice cold RPMI/5% FBS. The liver slurry was centrifuged at 300 g for 10 min at 4° C. in a 50 ml tube/liver. The pellet was resuspended in 10 ml of 0.02% collagenase IV (Sigma-Aldrich Corp.) in RPMI 1640 and left for 45 min at 37° C. 30 ml of ice cold RPMI/5% FBS was added to each tube, then centrifuged for 3 min at 30 g. Pellet was discarded. The supernatant was centrifuged for 10 min at 300 g at 4° C. The cell pellet was resuspended in 6 ml of ice cold RPMI 1640 (or in 45% Percoll (Amersham Biosciences, Uppsala, Sweden) and underlaid with 24% metrizamide (Sigma-Aldrich Corp.) in PBS (or with 70% Percoll, respectively). A centrifugation at 1000 g for 20 min at 4° C. followed. Lymphocytes at the interface were harvested, washed with RPMI/5% FBS and used for further analyses.

The degree of intrahepatic lymphocyte contamination by blood lymphocytes is likely minimal, as the results indicate a liver-specific increase in NK-T cells and a different ratio of N nucleotide insertions at the V-D and D-J junctionss in intrahepatic B lymphocytes (3.5 and 4.4) compared to blood B-cells (4.5 and 3.4, see also Results).

Isolation of Lymphocytes from Spleen, Blood and Peritoneal Cavity

Spleens were minced through a nylon mesh (Cell Strainer; BD Falcon, Bedford, Mass.) to obtain single cell suspensions in DMEM, 5% FCS, and 2 mM L-glutamine. Erythrocytes were lysed by incubating in lysis buffer (140 mM NH₄Cl, 17 mM Tris-HCl, pH 7.65) for 3 min on ice. Blood was collected into EDTA containing tubes (BD Pharmingen, San Diego, Calif.). To isolate blood lymphocytes, 200 μl of blood was underlaid with Ficoll-Paque (Amersham Biosciences, Uppsala, Sweden) and centrifuiged at 1000 g at RT for 20 min. Lymphocytes were collected from the interface. The peritoneal cavity (PC) was washed with 5 ml of DMEM, 5% FCS, and 2 mM L-glutamine to collect PC leukocytes. Following these procedures, lymphocytes were washed twice in DMEM, 5% FCS, and 2 mM L-glutamine by 300 g centrifiigation at 4° C. and resuspended in PBS/BSA/azide for flow cytometric analysis or in cell culture medium for proliferation studies.

Flow Cytometry

Fluorescence staining was performed as previously described. (Forster and Rajewsky, Eur. J. Immunol. 17:521-528 (1987)). Annexin V, 7AAD, and antibodies specific for IgM, IgD, CD19, CD23, CD5, CD69, CD86, B220, MHCII, CD43, Mac-1, CD4, CD8 (BD Pharmingen, San Diego Calif.) or CD21 (Ebioscience, San Diego, Calif.) were used. Antibodies were conjugated to FITC, PE, APC, PerCP, Cy-Chrome, or biotin. Biotinylated antibodies were detected with streptavidin conjugated to PerCP. Stained cells were fixed and analyzed using the FACScalibur (BD Biosciences, San Jose, Calif.).

In Vitro Stimulation of CFSE Labelled B-Cells

To generate a stock solution, CFSE (Molecular Probes, Eugene, Oreg.) was dissolved to 5mM in DMSO and stored at −80° C. Splenic B-cells were MACS purified by enrichment with MACS beads coupled to the anti-B220 Ab (Miltenyi Biotec, Auburn, Calif.) on the LS magnetic columns (Miltenyi Biotec) according to the manufacturer's instructions. Cells were then washed twice with RPMI 1640, resuspended at 5×10⁷ cells/ml in a 5 mM concentration of CSFE in warm RPMI 1640 for 10 min at 37° C. Cells were then washed 3 times in ice cold RPMI 1640/5% FCS, resuspended in RPMI 1640/5% FCS/\tilde{β}ME/L-glutamine at 2×10⁵/100 μl and transferred into a flat bottom 96 well plate in 100 μl/well). Another 100 μl RPMI were added that contained stimulating reagents at 2 times final concentration. The stimuli used were pure F(ab′)₂ fragment goat anti-mouse IgM (2.5 μg/ml; Jackson Immunoresearch, West Grove, Pa.), IL-4 (25 U/ml; R&D Systems, Minneapolis, Minn.), anti-mouse CD40 Ab (0.25 μg/ml, Ebioscience), anti-RP105Ab (10.5 μg/ml, Ebioscience), LPS (20 μg/ml, Sigma-Aldrich Corp.).

Immunohistochemistry

Antibody specific for alpha smooth muscle actin (clone 1A4, DakoCytomation, Carpinteria, Calif.) was used at 1:50 dilution with 30 min incubation. Heat induced epitope retrieval pretreatment of tissue sections was performed in 10 mM Citrate Buffer, pH 6.0 for 30 sec at 125° C., kept at 90° C. for 10 sec and cooled to RT for an additional 20 min prior to immunostaining. Binding of primary antibody to tissue elements was detected using an MM Biotinylation Kit (Biocare Medical, Walnut Creek, Calif.), with 3,3′-diaminobenzidine (DAB) substrate. Slides were counterstained with Mayer's Hematoxylin for 1 minute.

F4/80 specific antibody (clone CI:A3-1, Serotec Inc., Raleigh, N.C.), was used at a concentration of 20 μg/ml for 30 min. Tissue sections were pre-treated with Proteinase K (DakoCytomation, Glostrup, Denmark) for 5 min. at RT. Binding of primary antibody was detected using a Vector Elite ABC kit (Vector Laboratories, Burlingame, Calif.), using DAB substrate. Slides were counterstained with Mayer's Hematoxylin for 1 min.

TUNEL staining was performed using an ApopTag In Situ Apoptosis Detection kit (Chemicon International, Temecula, Calif.) according to the manufacturer's instructions. Labeled apoptotic cells were detected using DAB/nickel chloride as the substrate. Slides were counterstained for 5 min with Methyl Green (Vector Laboratories, Burlingame, Calif.).

Collagen fibers were detected using Sirius Red stain (Luna, Histopathologic Methods and Color Atlas of Special Stains and Tissue Artifacts: American HistoLabs, Incorporated. 767 pp. (1992)); H&E staining was performed as described elsewhere (Luna, Manual of Histologic Staining Methods of the Armed Forces Institute of Pathology. New York: McGraw-Hill Book Company (1968)).

PCR and Ig Gene Rearrangement Analysis

DNA was extracted from cells positively selected on CD19⁺ magnetic beads (Miltenyi Biotec,) according to Genomic DNA isolation kit (Qiagen, Valencia, Calif.) manufacturer's protocol. DNA (2 μl, equivalent of about 10³ B-cells) was used for amplification of the VDJ joints. Two rounds of amplification were performed using VHA, VHB and VHE 5′ primers specific for J558L, Q52 and 7183 V_{H} families and JH4E 3′ primer (16) for the first and nested JH1 or JH4A 3′ primers for second rounds. All primers were synthesized at Biogen Idec. Twenty cycles were performed for the first round (1 min at 95° C., 1 min at 60° C., and 1.5 min at 72° C.); 30 cycles (1 min at 95° C., 1 min at 63° C., and 1.5 min at 72° C.) were done for the second round, using 2 μl of the first round reaction as a template. The expected 0.4 kb fragment was purified from the gel and subcloned into the pCR4-TOPO vector (Invitrogen, Carlsbad, Calif.). DNA from individual colonies was prepared and sequenced using standard vector specific primers.

Interstitial Collagen Quantification

A total of 3 sections from a liver (each from a different lobe) were stained from each animal. Black and white pictures of Sirius red staining were made in polarized light at 5× magnification. Pictures were made such that liver tissue occupied the whole area captured by the camera to ensure that total image area was identical in each picture (4-10 pictures per animal). Vessels constitutively containing collagen were electronically removed from each image. Next the amount of white staining (interstitial collagen) was quantified by MetaMorph image analysis software (Universal Imaging Corporation, Downingtown, Pa.). Quantification is displayed in arbitrary units (1 correlates to 1000 pixels). The absolute amount of white area cannot be directly compared between different experiments, because it varied with the intensity of Sirius Red staining.

Results

B-Cells Represent a Major Lymphocyte Population in the Liver

B-cells have been extensively studied in embryonic liver, the major site of hematopoiesis in the developing embryo. However, little is known about hepatic B-cells in the adult liver. In this Example, intrahepatic (IH) B-cells were phenotypically and functionally characterized.

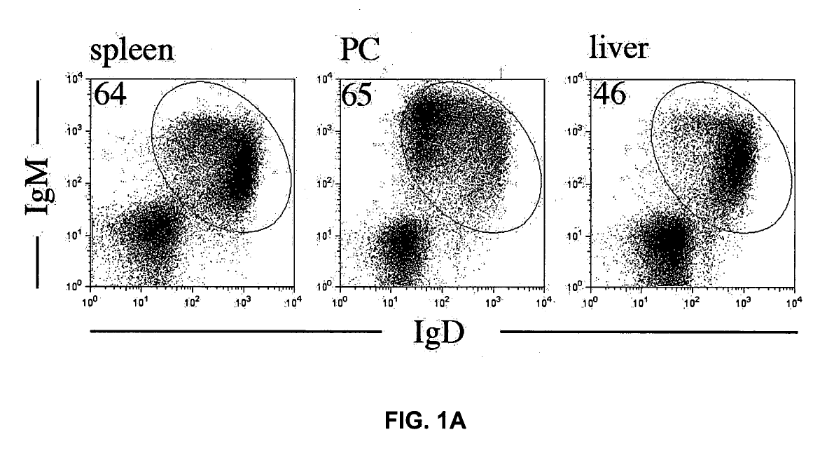

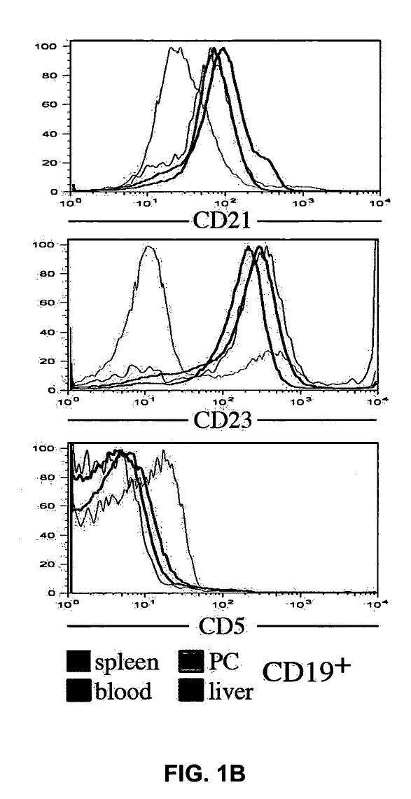

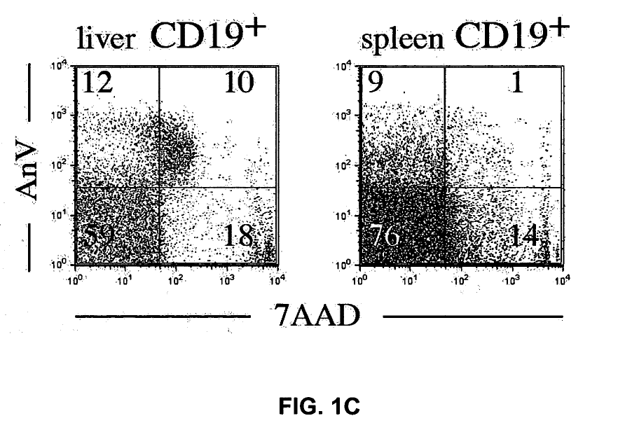

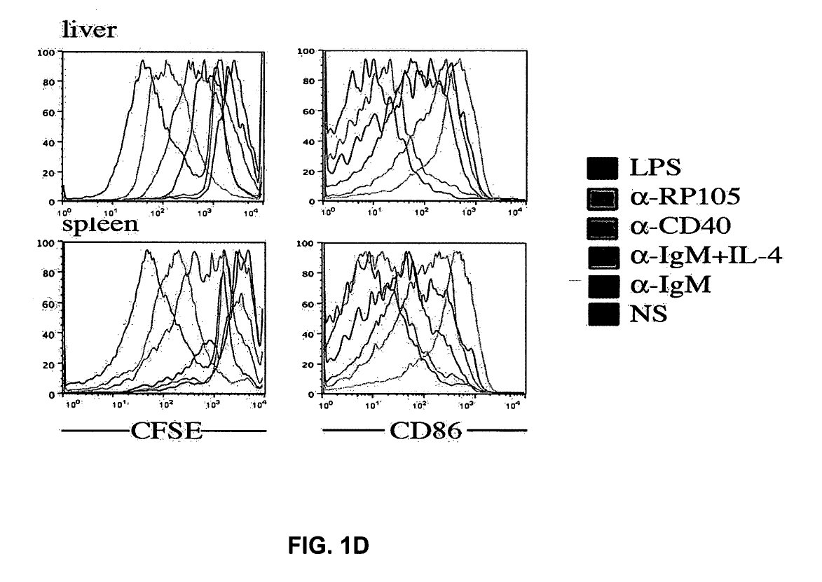

After enriching the lymphocyte population from PBS-perfused liver, the proportion of IHB-cells was quantified by staining for CD19, a B lineage specific marker. In both BALB/c and C57BL/6 mice, B-cells represent about 50% of IH lymphocytes (range 30-60%, FIG. 1A and data not shown). The absolute number of B-cells isolated from a liver was ˜2×10⁶. CD19⁺ IHB-cells were shown to express IgM, IgD, B220, MHCII, and CD62L at levels similar to their splenic counterparts (FIG. 1A and B and data not shown). IHB-cells do not express the CD43 and Mac-1 markers typical for B-1 or immature B-cells (data not shown). IHB-cells express CD5 at a level higher than that detected on blood B-cells, but lower than observed on PC B-cells (FIG. 1B). Higher CD5 levels are indicative of conventional B-cell activation. (Cong et al., Int. Immunol. 3:467-476 (1991)). IHB-cells express CD23, but at a lower level than splenic or blood B-cells. CD21 surface expression is also slightly lower for IHB than for splenic B-cells, but higher than for blood B-cells (FIG. 1B). Taken together, with regard to expression of these markers, liver B-cells are most similar to follicular splenic B-cells.

Hepatic B-Cells are Functionally Competent

As liver is often regarded as a destination for dying lymphocytes (Crispe et al., Immunol. Rev. 1 74:47-62 (2000)), it was determined whether IHB-cells are pro-apoptotic using Annexin V which binds to phospholipid phosphatidylserine (PS) that translocates from the inner to the outer layer of the cellular membrane as cells undergo apoptosis. Annexin V bound up to 30% of hepatic B-cells compared to ˜15% of splenic B-cells (FIG. 1C and data not shown). Thus, most liver B-cells do not show a predisposition to apoptosis, and the higher number of apoptotic cells in liver compared to spleen might be related to differences in lymphocyte isolation.

The proliferative capacity of B lymphocytes in response to mitogenic and B-cell receptor crosslinking is an important functional characteristic which differs substantially for B-cell subsets. (Morris and Rothstein, J. Exp. Med. 1 77:857-861 (1993); Philips et al., Immunol. Cell. Biol. 76:332-342 (1998); Erickson et al., 2001. J. Immunol. 166:1531-1539 (2001)). Hepatic and splenic B-cells were compared for their extent of proliferation and upregulation of costimulatory molecules, such as CD86 (B7.2) and MHCII, in response to various stimuli. Interestingly, the proliferative response of IHB-cells was very similar to that of splenic B lymphocytes (FIG. 1D): the response to Toll-like receptor 4, RP105 and CD40 stimulation is the same, whereas response to IgM crosslinking is greater in the absence, but not in the presence of IL-4. The greater proliferative response upon IgM crosslinking only may reflect better survival of IHB-cells in culture without an exogenous survival factor like IL-4, and is consistent with an activated status of IHB-cells suggested by CD5 upregulation (FIG. 1B). The extent of upregulation of MHCII, CD86 and CD5 by all stimuli tested was very similar for liver and splenic B-cells (FIGS. 1B, D and data not shown).

IHB-Cells Resemble Splenic B2 Cells and are not of Embryonic Liver Origin

B-cells in adult liver may represent residual hepatic B-cell generation from embryonic liver. Alternatively, IHB-cells may be bone marrow (BM) derived as are splenic B-cells in an adult organism. To address the origin of intrahepatic B-cells, genetic analyses of their VDJ rearrangements were performed. Few insertions of non-templated (N, P) nucleotides are seen in the VDJ junctions of neonatal B-cells generated in the embryonic liver, similar to what has been reported for B1 cells. (Feeney, J. Exp. Med. 172:1377-1390 (1990); Gu et al., EMBO J. 9:2133-2140 (1990); Meek, Science 250:820-823 (1990)). In contrast, adult splenic and blood B-cells have extensive non-templated nucleotide additions. (Kantor et al., J. Immunol. 158:1175-1186 (1997); Kepler et al., J. Immunol. 157:4451-4457 (1996)). CDR3 sequences derived from pooled adult liver lymphocytes were compared to those derived from splenic cells of 2 day old mice or adult mouse blood B-cells. Adult IHB-cells markedly differ from neonatal B-cells and resemble splenic B2 cells or recirculating blood B-cells in their VDJ joint sequence. The average number of N, P nucleotides in neonatal B-cells is 0.5 for the VD junction and 0.1 for the DJ junction. This is notably different from 3.5 (or 4.5) for the VD and 4.4 (or 3.4) for the DJ junctions of B-cells in the adult liver (or blood). Interestingly, adult liver and blood B-cells also appear different in the length of their VD and DJ junctions; this difference is on the border of being statistically significant, p=0.1, student's t-test. IHB-cells have fewer N, P nucleotides in their VD joint than in their DJ joint, the converse of what is reported for conventional adult B2 cells. (Kantor et al., J. Immunol. 158:1175-1186 (1997)). The difference in the length of N,P insertions in the IHB and adult blood B-cells might be a result of intrahepatic B-cell selection. In addition, the difference strengthens the notion that liver B-cells represent a true intrahepatic population with no significant contamination by peripheral blood B-cells.

B-Cell Role in Hepatic Fibrosis

To assess the physiological role that B-cells might play in liver, liver disease was induced and disease progression compared in mice lacking B-cells with WT animals. The CCl₄ induced liver injury model was used, in which a pronounced necroinflammatory liver injury, occurring with every CCl₄ administration, is followed by a chronic repair response. This model was considered to have an advantage over many widely utilized liver injury models (e.g. schistosome, LPS, ConA) because the toxic insult induces general hepatotoxicity, rather than a priori targeting a defined part of the immune system. Yet, interestingly, it was found that B-cells in the liver are particularly sensitive to CCl₄ application. IHB-cell numbers drop approximately 10-fold 1 day after a CCl₄ treatment as opposed to other intrahepatic lymphocytes (NK-T, T cells), which remain unaffected at this time point (data not shown). By day 5 after a CCl₄ injection, B-cell numbers recover (data not shown).

To test whether B-cells have a role in liver injury and repair, B-cell deficient mice in CCl₄ induced hepatotoxicity studies were used. The B-cell deficient mouse strain chosen for analysis carries a targeted deletion in the J_{H} region of the immunoglobulin heavy chain gene, which precludes assembly of a coding heavy chain gene and, thus, prevents B-cell and antibody generation. (Chen et al., Int. Immunol. 5:647-656 (1993)). These B-cell deficient mice are referred to herein as J_{H}−/− mice.

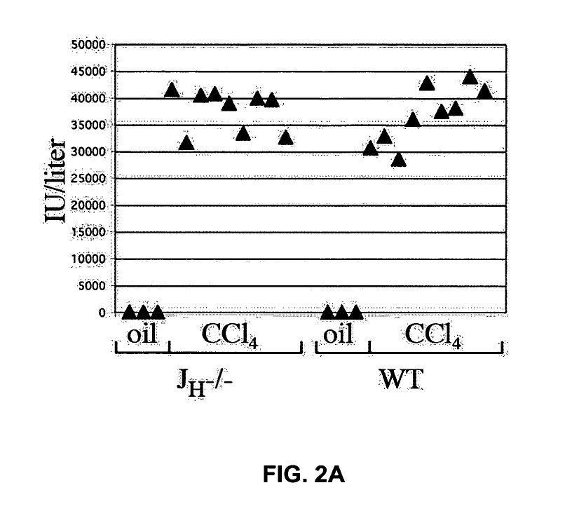

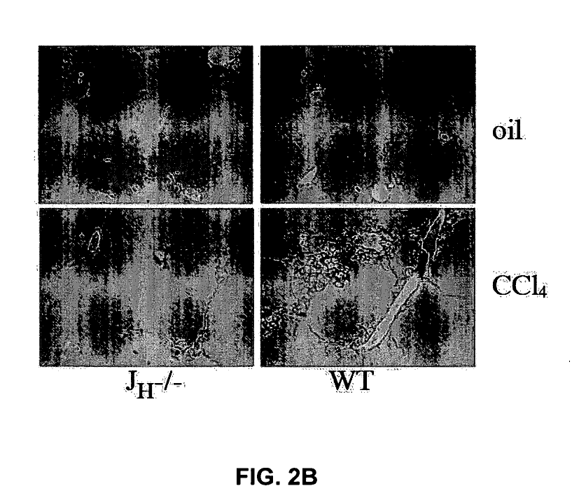

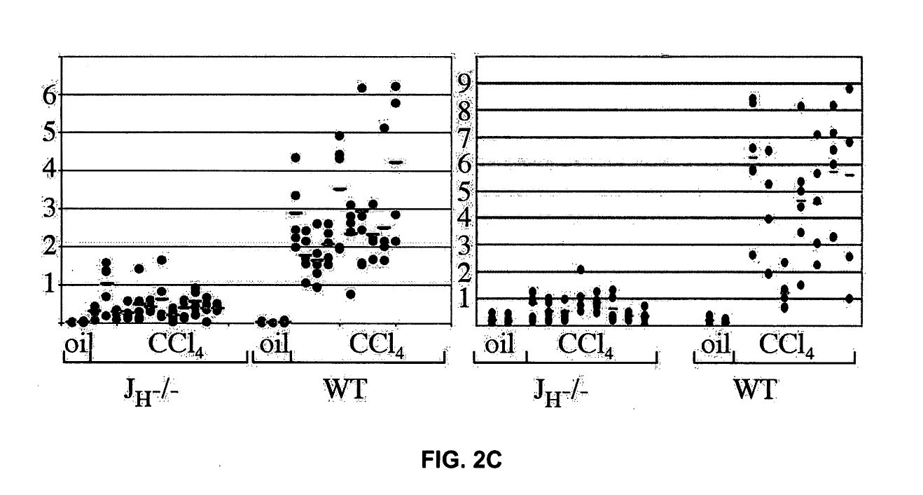

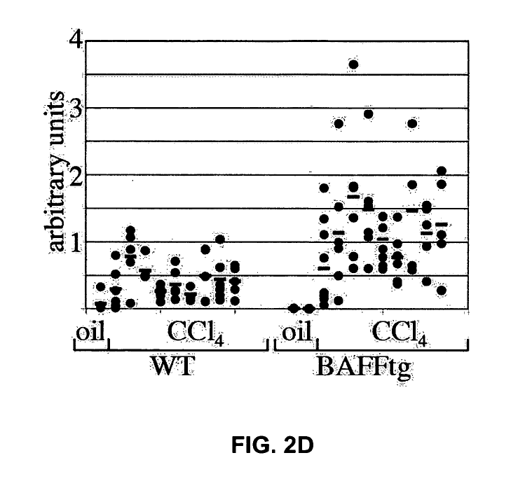

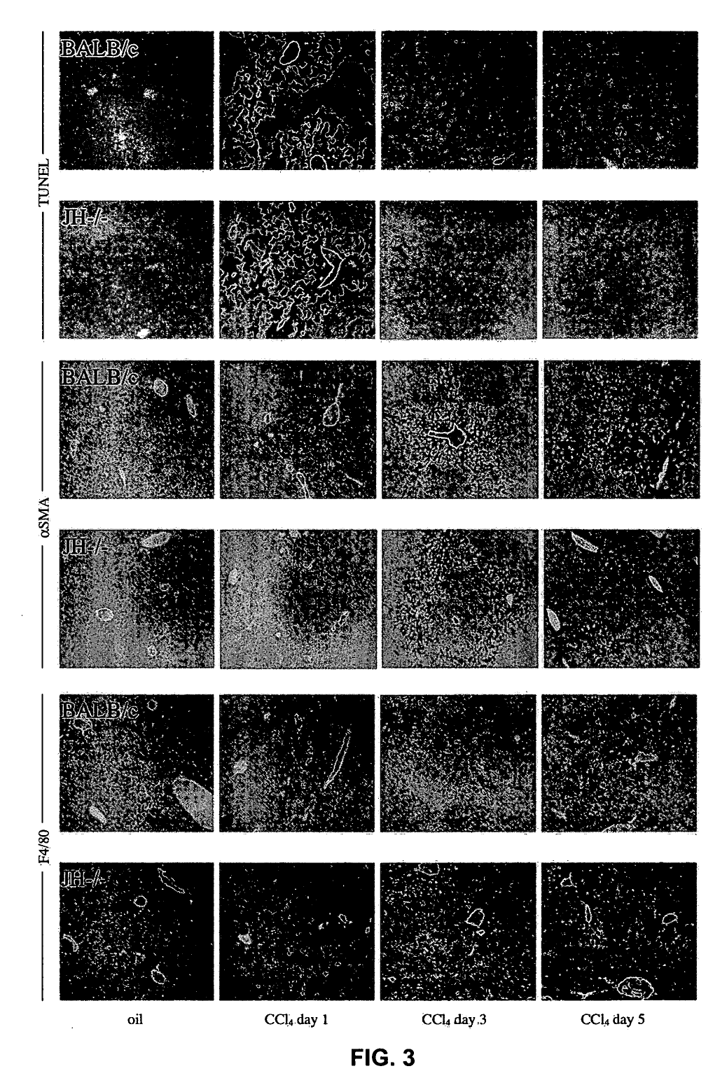

The extent of CCl₄ induced hepatocyte injury, assessed by the release of the hepatocyte specific enzyme ALT into serum 24 hours after a CCl₄ treatment, was similar in J_{H}−/− and WT BALB/c mice (FIG. 2A). It is also obvious from histological analysis (FIG. 3 and see below). Interestingly, however, there was a large difference in the amount of collagen fibers accumulating; J_{H}−/− mice had about 6-8 fold less interstitial collagen deposition compared to WT mice one week after the sixth weekly dose of either 1.75 or 3.5 mg/kg CCl₄ (FIGS. 2B and 2C). No significant changes in the number or location of F4/80⁺ macrophages and smooth muscle actin producing myofibrobasts were observed after 6 CCl₄ treatments (data not shown). Thus, B-cells appear to constitute a non-redundant cell population necessary for the liver to develop fibrotic changes in response to CCl₄.

To test whether B-cell function is limited to the specific case of CCl₄ induced injury or, rather, plays a more general role in hepatic tissue repair, hepatotoxicity was induced with 1-naphthylisothiocyanate (ANIT), as ANIT causes liver destruction by a mechanism distinct from that induced by CCl₄. The hepatotoxicity induced by ANIT is manifested as neutrophil-dependent necrosis of bile duct epithelial cells and hepatic parenchymal cells. (Hill et al., Toxicol. Sci. 47:118-125 (1999)). After 8 weeks of ANIT treatment, it was found that J_{H}−/− had about 7 times less collagen deposits than WT mice. Thus, fibrosis is reduced in the absence of B-cells in at least two model systems.

To examine whether increasing B-cell numbers above normal leads to more pronounced fibrosis, BAFF-tg mice that show a 20-30% increase in B-cell numbers (Mackay et al., J. Exp. Med. 190:1697-1710(1999)) compared to the corresponding C57B1/6 WT control mice were used. Following six CCl₄ treatments, fibrosis developed in the BAFF-tg and C57B1/6 controls. This fibrosis was characterized by less collagen fiber deposition than noted in BALB/c mice (data not shown and Shi et al., Proc. Natl. Acad. Sci USA 94:10663-10668 (1997)). Interestingly however, the BAFF transgenic mice had about twice the amount of collagen deposits as their WT C57B1/6 counterparts (FIG. 2D).

B-Cell Deficient and WT Mice Respond Differently to a Single CCl₄ Induced Injury

To understand what acute effects trigger changes in collagen deposition after 6 weeks of treatment, the kinetics of tissue changes were analyzed in liver sections of B-cell deficient and control mice 1, 3 and 5 days post a single CCl₄ challenge. Interestingly, TUNEL staining, detecting apoptotic cells, showed that despite similar initial injury at day 1, J_{H}−/− mice clear apoptotic cells completely by day 3, whereas in WT mice some dying cells are still detected even 5 days post injury (FIG. 3). When staining sections for the tissue macrophage specific marker F4/80, it was found that as early as day 1, there is a small increase in macrophage numbers in J_{H}−/− compared to WT mice, which becomes very substantial by days 3 and 5 (FIG. 3). Thus, it seems that in the absence of B-cells, macrophages are better able to clear dying hepatocytes. As the major cellular source for collagen fibers is a population of myofibroblasts (Rockey et al., Clin. Liver. Dis. 4:319-355 (2000)), smooth muscle actin that marks myofibroblasts in the injured liver was also monitored. Myofibroblasts are first detectable at day 3, at similar levels in B-cell deficient and control mice. By day 5, however, WT mice show many more myofibroblasts (FIG. 3). With repeated injury, the inability of macrophages to efficiently remove dying hepatocytes may lead to over-stimulation of myofibroblasts and eventually result in the greater deposition of collagen noted upon long term injury. In a recent study (Duffield et al., J. Clin. Invest. 115:56-65 (2005)), macrophages were shown to play distinct, opposing roles during liver injury and repair. It appears that in the absence of B-cells, those macrophages that contribute to recovery from inflammatory scarring are preferentially activated.

CD4⁺, CD8⁺ or γδ T Cells do not Influence Hepatic Fibrosis to a Significant Degree

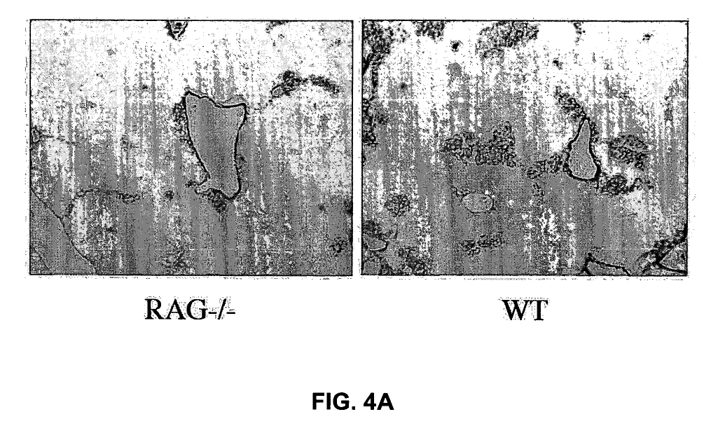

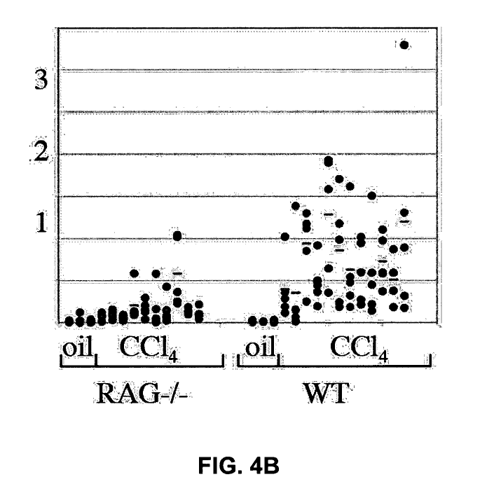

To assess whether mice deficient in T cells also have a defect in fibrogenesis, a series of CCl₄ induced liver injury experiments was performed with mice that lack both B and T cells (RAG2−/−), CD4⁺ T cells (Aβ−/−), CD8⁺ T cells (β2m−/−), or γδT cells (TCR δ−/−). For every mouse mutant strain, a control strain of the same genetic background was used (see Materials and Methods). Of these, only RAG2−/− mice showed dissimilar amount of collagen deposition following long term treatment with CCl₄ compared to appropriate WT counterparts (FIG. 4 and data not shown). RAG2−/− mice, lacking all lymphocytes that require DNA rearrangement to assemble their receptors, show approximately a 3-4 fold reduction in interstitial collagen accumulation compared to WT mice (FIG. 4B). This result is very similar to the result obtained in mice lacking only B-cells, and does not imply a prominent role for T cells in the CCl₄ model of liver fibrosis.

B-Cell Role in Liver Fibrosis is Antibody-Independent

B-cells can mediate local effects such as antigen presentation, cytokine release, and/or cell-cell contact regulated by co-stimulatory molecules, and long range effects via antibodies. As T cell deficient animals (see above) did not show any differences in collagen deposition, B-cell antigen presentation to T cells is unlikely to influence liver fibrosis.

To determine whether B-cell regulation of liver fibrosis requires immunoglobulin, two mouse strains that have normal numbers of B-cells but either lack Ig in their serum or have Ig levels severely reduced were used. Mice expressing Epstein-Barr virus derived protein LMP2a from a gene incorporated at the place of J elements of the IgH locus (D_{H}LMP2a allele (Casola et al., Nat. Immunol. 5:317-327 (2004)) lack both surface and circulating immunoglobulin, whereas mice expressing a mIgM transgene on the J_{H}−/− background encode surface, but not secreted Ig. (Chan et al., J. Exp. Med. 189:1639-1648 (1999)).

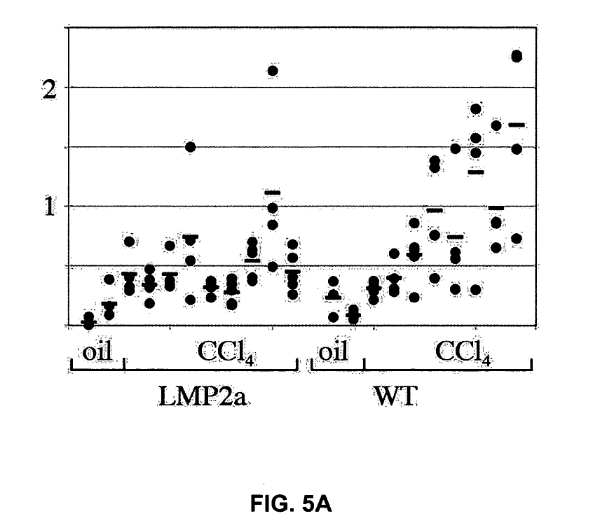

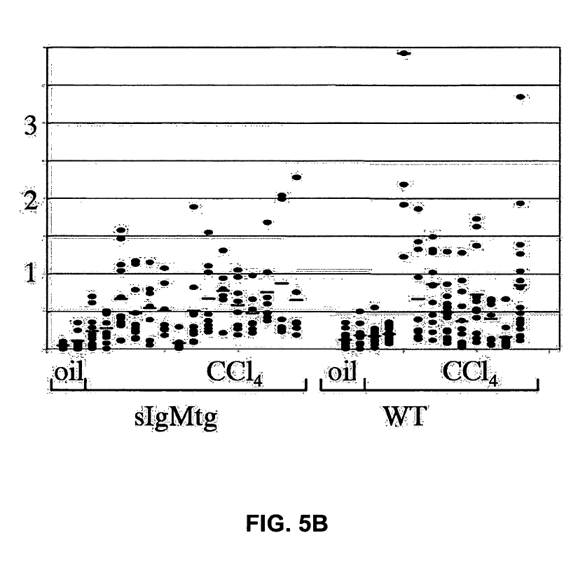

As shown in FIG. 5A, following 6 weekly treatments of 1.75 mg/kg CCl₄, similar levels of collagen deposition were noted in mice expressing Epstein-Barr virus derived LMP2a protein and their WT BALB/cAnNCrlBr controls. Moreover, mIgM tg (J_{H}−/−) mice expressing surface, but not secreted Ig (Chan et al., J. Exp. Med. 189:1639-1648 (1999)), showed the same degree of CCl₄ induced liver fibrosis as WT control BALB/c mice (FIG. 5B). Thus, B-cell effects on the pathology of CCl₄ induced liver fibrosis are antibody independent. It is noteworthy that the degree of fibrosis in WT BALB/c mice in these experiments is lower than in previous ones (FIGS. 2, 4, 5) potentially because of different housing conditions and/or concurrent infection of these animals: Both LMP2a and mIgM mouse colonies were positive for H. hepaticus; therefore, these mice as well as corresponding WT strains were kept in the quarantine facilities.

Discussion

In this Example, it is demonstrated that intrahepatic B-cells represent a sizable population with phenotypic and functional characteristics resembling that of conventional B2 cells. IHB-cell express CD5 to somewhat higher degree that conventional B2 cells and they proliferate better in response to IgM crosslinking without supplementing IL-4 in vitro (FIG. 1), implying activated status of IHB-cells. Despite the fact that adult liver has been known to contain c-kit⁺ pluripotent hematopoietic stem cells that could give rise to multilineage leukocytes (Watanabe et al., J. Exp. Med. 184:687-693 (1996); Taniguchi et al., Nat. Med. 2:198-203 (1996)), most B-cells in the adult liver appear to be BM-derived in contrast to self-propagating embryonic liver derived B1 lineage cells. (Herzenberg, Immunol. Rev. 1 75:9-22 (2000)). IHB-cells are likely of the BM origin: VDJ junctions of intrahepatic B-cells contain extensive N nucleotide insertions, with similar total average length to conventional B2 cells. Notably, expression of terminal deoxyribonucleotidyl transferase (TdT), the enzyme responsible for N nucletide insertion, has not been studied in adult liver (Benedict et al., Immunol. Rev. 175:150-157 (2000)), thus, the formal but unlikely possibility exists that adult liver B-cells are generated in the liver in a TdT-dependent fashion.

In this Example it is shown that B-cells play an important antibody-independent role in the development of liver fibrosis, adding another disease model likely dependent on local B-cell function. An imperative role of B-cells has also been demonstrated for autoimmune diabetes in nonobese diabetic (NOD) mice. B-cell-deficient NOD.Igμnull and B-cell-depleted NOD mice did not develop insulitis or insulin-dependent diabetes mellitus, supporting the idea that B-cells are critical for the initiation and/or activation of autoreactive T cells. (Serreze et al., J. Exp. Med. 184:2049-2053 (1996); Noorchashm et al., Diabetes 46:941-946 (1997)). B-cells were also shown to be required for lupus nephritis in the polygenic, fas-intact and fas-deficient MRL model of systemic autoimmunity. (Chan et al., J. Exp. Med. 189:1639-1648 (1999); Chan et al., J. Immunol. 160:51-59 (1998); Chan et al., J. Immunol. 163:3592-3596 (1999)). In both cases, an antibody-independent mechanism turned out to be crucial for B-cell involvement. (Chan et al., J. Exp. Med. 189:1639-1648 (1999); Wong et al., Diabetes 53:2581-2587 (2004))

In this Example, mice have been used that are constitutively devoid of B-cells to study B-cell involvement in fibrotic pathology. Although normal in gross physiology, B-cell deficient mice lack follicular dendritic networks (Fu et al., J. Exp. Med. 187:1009-1018 (1998); Gonzalez et al., J. Exp. Med. 187:997-1007 (1998); Endres et al., J. Exp. Med. 189:159-168 (1999)), follicle associated epithelium in the intestinal Peyer's patches (Golovkina et al., Science 286:1965-1968 (1999)), and a non-canonical subset of NK-T cells. (Treiner et al., Nature 422:164-169 (2003)). B-cell-less mice also have defects in CD4⁺ T cell function (Baumgarth et al., Proc. Natl. Acad. Sci. USA 97:4766-4771 (2000)), and perhaps some other as yet undescribed developmental/functional deficiencies. Thus, the results obtained with the B-cell deficient mice indicate that B-cells indirectly affect the pathogenesis of liver fibrosis.

Since T cell deficient mice do not show any difference in the development of liver fibrosis (data not shown), a CD4⁺0 cell defect is unlikely to account for the strongly attenuated liver fibrosis observed in the J_{H}−/− mice. However, a B-cell dependent NK-T cell subset that expresses Vα19 containing invariant TCR (Treiner et al., Nature 422:164-169 (2003)), resident in murine liver (Shimamura et al., FEBS Lett. 516:97-100 (2002)), might contribute to the reduced fibrosis noted in the B-cell deficient mice. NK-T cells are known for their ability to respond in a rapid manner and to produce both TH1 and TH2 type cytokines. (Godfrey et al., J. Clin. Invest. 114:1379-1388 (2004)). Such qualities allow NK-T cells to participate in immune response regulation. (Godfrey et al., J. Clin. Invest. 114:1379-1388 (2004)). No pronounced differences in liver fibrosis development in CD1−/− mice (data not shown) that lack conventional Vα14 TCR NK-T cells was found. Unfortunately, there is no mouse mutant available that allows one to address the role of non-canonical Vα19 invariant NK-T cells in liver fibrosis. Nonetheless, as RAG−/− animals show inhibition of fibrosis to a similar extent as B-cell deficient mice, a role for cell types that require gene rearrangement for their development (B and T cells of various lineages) is implied. Thus, together the data suggest that either non-CD1 restricted NK-T cells that require B-cells for their development (Treiner et al., Nature 422:164-169 (2003)) or B-cell autonomous function has a role in the fibrosis manifested in the CCl₄ induced hepatotoxicity model.

By using two previously generated mouse strains (LMP2a insertion and mIgM-Tg mice) deficient in immunoglobulin production, it was shown that antibodies are not required to develop CCl₄ induced liver fibrosis. LMP2a mice have normal B-cell numbers and completely lack both secreted antibodies and surface expression of immunoglobulin. (Casola et al., Nat. Immunol. 5:317-327 (2004)). LMP2A does not only mimic BCR signaling, but triggers additional signaling pathways (Ikeda et al., J. Virol. 77:5529-5534 (2003); Portis and Longnecker, J. Virol. 77:105-114 (2003)), thus, fibrogenesis in the mIgM-Tg (J_{H}−/− ) mice that express a transgenic surface BCR and have 300-500 fold reduced antibody titers compared to normal mice (Chan et al., J. Exp. Med. 189:1639-1648 (1999)) was assessed. Both mouse lines developed liver fibrosis to an extent similar to controls. Thus, the B-cell role in liver fibrosis pathology appears to be antibody-independent, suggesting that it is mediated by functions (e.g. cytokine secretion and/or cell-cell contact) of local B-cells as opposed to potentially long-range effects mediated by B-cells localized elsewhere in the organism. An antigen presentation role for B-cells is unlikely to play a significant role in liver fibrosis, as mice deficient in conventional T cells show similar fibrogenesis as their WT counterparts. Moreover, LMP2a B-cells do not have the ability to bind, internalize and present antigens, because they lack B-cell receptor on the surface and also show similar collagen deposits to WT mice. Together these data suggest that liver tissue repair is affected by local B-cell function, which may be mediated in part by the IHB-cells defined herein. Formally it is possible that B-cells overwhelm clearance mechanism(s) in the liver. However, B-cell numbers are very small compared to hepatocyte numbers.

The results presented in this Example are in agreement with reports that the degree of hepatic damage in response to CCl₄ was significantly milder in splenectomized compared to sham-operated rats (Chen et al., Chin. Med. J. (Engl) 111:779-783 (1998)) and in SCID mice on BALB/c background compared to appropriate controls. (Shi et al., Proc. Natl. Acad. Sci. USA 94:10663-10668 (1997)). However, liver fibrosis induced by the Schistosoma mansoni parasite is increased in B-cell deficient compared to control mice. (Ferru et al., Scand. J Immunol. 48:233-240 (1998)). The differences in the fibrosis induction mechanism by repeated hepatocyte damage (as is the case for CCl₄ and ANIT) or by low level worm infection could explain the discrepancy.

B-cell function has also been associated with fibrosis in the skin in both mice and humans. In the tight-skin (TSK/+) mouse as well as in systemic sclerosis patients, chronic B-cell activation resulting from augmented CD19 expression leads to skin fibrosis and autoimmunity. (Saito et al., J. Clin. Invest. 109:1453-1462 (2002)). Moreover, a B-cell line established from the lung tissue of a patient with scleroderma exhibits augmented proliferation and inflammatory response that are likely to lead to fibrotic changes. (Kondo et al., Cytokine 13:220-226 (2001)).

In sum, this Example describes the isolation and characterization of adult liver B-cell population and directly demonstrates a role for B-cells in tissue repair following hepatic injury. This Example further demonstrates that B-cells are involved in the pathology of fibrosis conditions. Thus, the results presented here indicate that B-cell antagonists may prove to be effective in treating fibrosis conditions.

### Example 2

### Pulmonary Fibrosis in a B-Cell Deficient Mouse Model

Introduction

Pulmonary fibrosis can be induced in animal models by exposure to bleomycin. The intratracheal administration of bleomycin in rodents is the most widely used model of lung fibrosis. Bleomycin is a cytotoxic agent that causes endothelial and epithelial injury, in part via generation of free radicals and induction of inflammatory cytokines. (Sleijfer, Chest 120:617-624 (2001)). Fibroblasts are activated, and by two weeks, there is significant fibrosis and collagen deposition in the lung. In this Example, it is shown that B-cell deficient mice, after sustained systemic exposure to bleomycin, exhibited enhanced survival and reduced lung fibrosis as compared to wild-type mice treated identically.

Materials And Methods

Mice

C57BL/6J: wild-type mice with normal B-cell function;

B6.129S2-lgh-6^{tm1Cgn}/J: B-cell deficient mice.

Sustained Bleomycin Exposure

On day 0, wild-type or B-cell deficient mice were aseptically implanted with a subcutaneous 7-day Alzet® osmotic minipump containing saline (n=7, wt; n=5, ko) or bleomycin solution at dosage levels of 60 (n=12, wt; n=10, ko), or 100 (n=8, wt; n=10, ko) mg/kg (total dose delivered over 7 days).

Measurements

Body weight and clinical signs were monitored for a 1-month period. Mice were euthanized on Day 28 and lungs removed, instilled with, and fixed in 10% neutral buffered formalin. Lungs were stained with Masson's trichrome to identify existing collagen/fibrosis, and immunohistochemically for α-actin to identify the degree of potential for future fibrosis. Proportion of tissue area occupied by collagen or actin was determined histomorphometrically using Metamorph® software.

Results

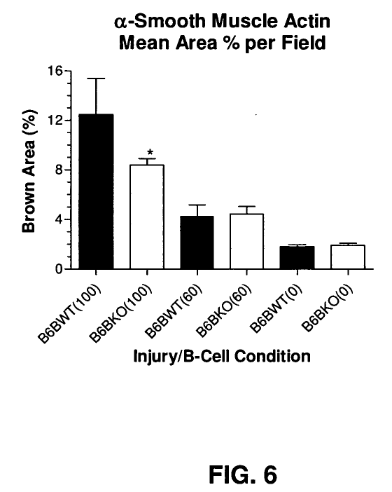

Administration of 60 mg/kg/7d bleomycin produced only modest α-actin accumulation by day 28. Wild-type and B-cell knockout mice showed similar α-actin levels. (FIG. 6).

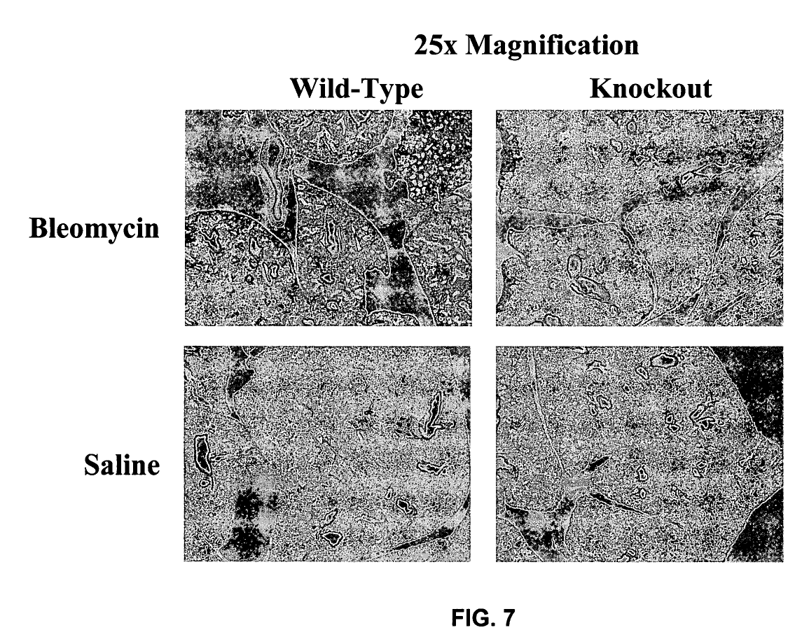

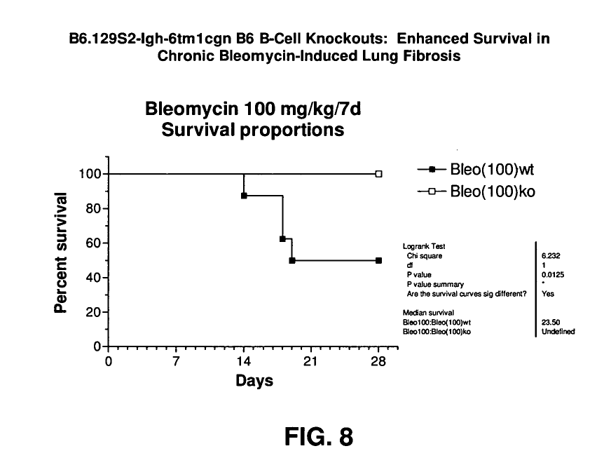

Administration of 100 mg/kg/7d bleomycin produced moderate to extensive α-actin accumulation by day 28. Wild-type animals showed reduced survival and statistically greater α-actin levels than B-cell knockout mice. (FIGS. 6, 7 and 8).

The results of these experiments demonstrate that the absence of B-lymphocytes reduces the extent of pulmonary fibrosis and enhances survival after sustained bleomycin exposure in C57BL6 mice. Accordingly, these results further support the use of B-cell antagonists for treating fibrosis conditions and, in particular, fibrotic conditions of the pulmonary system.

### Example 3

### Kidney Fibrosis in A B-Cell Deficient Mouse Model

Introduction

Unilateral ureteral obstruction (UUO) is a model of obstructive nephropathy the produces progressive tissue compression, tubular degeneration, and interstitial and glomerular fibrosis. (Miyajima et al., Kidney International 58:2301-2313 (2000)). In this Example, it is shown that B-cell deficient mice exhibited reduced renal fibrosis in response to UUO as compared to wild-type mice.

Materials And Methods

Mice

C57BL/6J: wild-type mice with normal B-cell function;

B6.129S2-lgh-6^{tm1Cgn}/J: B-cell deficient mice.

Unilateral Ureteral Obstruction

On Day 0, the left ureter was isolated, ligated and sectioned between ligatures in wild-type (n=10) or B-cell deficient (n=10) mice, aseptically under ketamine/xylazine anesthesia. Unoperated wild-type (n=5) or B-cell deficient (n=5) mice were also included as normal controls.

Measurements

Body weight and clinical signs were monitored during the 10-day progression to peak disease. Mice were euthanized on Day 10 and both kidneys removed, and fixed in 10% neutral buffered formalin. Kidneys were stained with Masson's trichrome to identify existing collagen/fibrosis, and immunohistochemically for α-actin to identify the degree of potential for future fibrosis. Proportion of tissue area occupied by collagen or actin was determined histomorphometrically using Metamorph® software.

Results

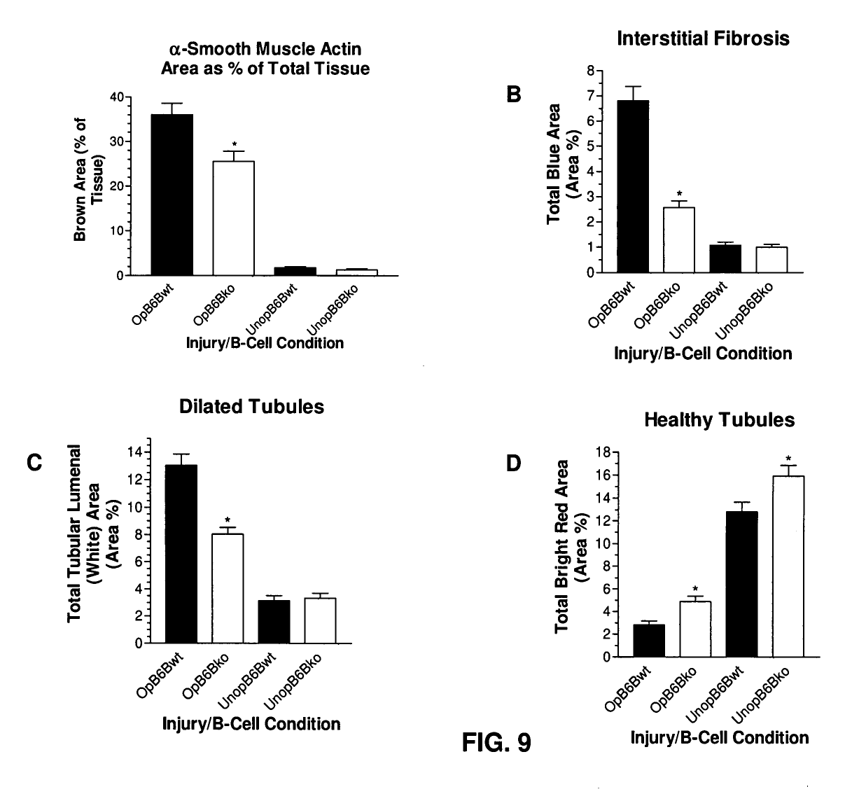

Following UUO, B-cell deficient mice exhibited a statistically significant 29% reduction in a-actin staining compared to wild-type counterparts (FIG. 9A) and a statistically significant 62% reduction in accumulated interstitial collagen (FIG. 9B). It will be appreciated that these pathological conditions constitute two classical markers for the measurement and quantification of fibrotic conditions.

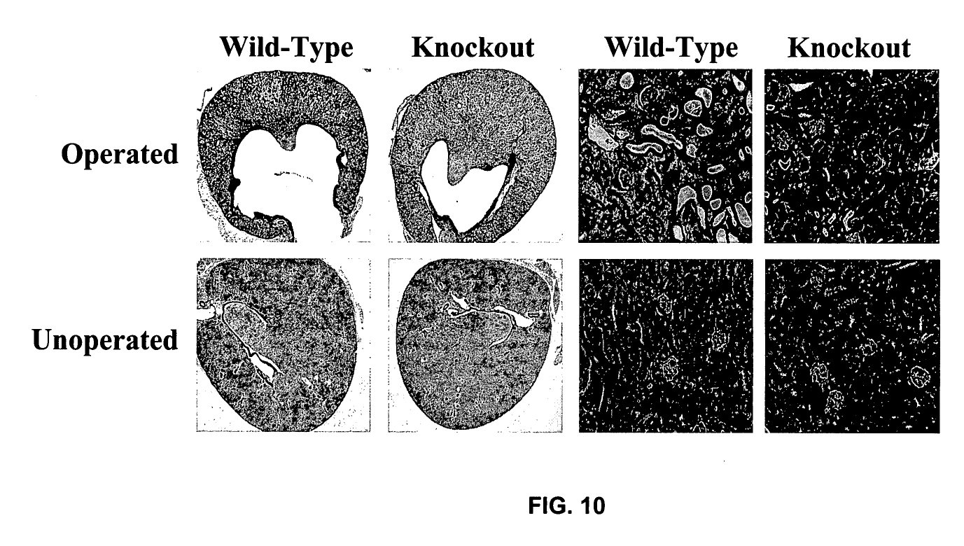

Following UUO, B-cell deficient mice also exhibited significantly reduced tubular dilation (FIG. 9C) and significantly increased healthy tubule staining (FIG. 9D). Increased healthy tubule staining was observed in B-cell deficient mice even in the absence of injury (FIG. 9D; see also FIG. 10).

The results of these experiments demonstrate that the absence of B-lymphocytes reduces the extent of renal fibrosis after UUO-induced injury.

The observation that the extent of experimentally-induced fibrosis injury in multiple model systems is substantially reduced in mice that are B-cell deficient (see Examples 1-3) strongly supports the use of B-cell antagonists to treat a variety of disease indications associated with inflammatory/fibrotic pathology.

### Example 4

### An Anti-CD20 Monoclonal Antibody Counters the Increase in Lung and Splenic B-Cells Caused by Bleomycin Treatment

Introduction

As demonstrated in Example 2, pulmonary fibrosis is induced in animal models by exposure to bleomycin, and the extent of bleomycin-induced pulmonary fibrosis is reduced in B-cell deficient mice. In this Example, it is shown that mice treated with bleomycin exhibit an increase in B-cells in their lungs, and importantly, this bleomycin-induced increase in B-cells is significantly reduced in mice that are treated with an anti-CD20 antibody. These results provide additional support for the use of B-cell antagonists to treat fibrosis conditions.

Materials and Methods

In this example, C57B1/6 male mice 9 weeks of age were used for the experiments. Day 0 mice were anesthetized with Ketamine/Xylazine IP, and given 0.025 units in 50 μl volume IT of Bleomycin using a PennCenntury Aerosolizer. The PennCenntury Aerosolizer was inserted through the mouth and into the trachea. The mice were administered either anti-murine CD-20 monoclonal antibody (designated “18B12,” developed at Biogen Idec, U.S. Appl. No. 60/741,491), or PBS, intraperitoneally on days −7 (7 days prior to administering bleomycin) and on day 7. A separate group of mice received only PBS intrachacheally and no other treatment.

Animals were euthanized by CO₂ on Day 9 and lungs and spleens were collected. Lungs and spleens were cut into segments with scissors, then homogenized and transferred to a 50-ml centrifuge tube. 40 ml ice-cold RPMI 1640/5% FBS was added to the tube and centrifuged for 10 min at 300×g (1200 rpm in IEC Centra 8R with standard rotor), at 4° C. to sediment and deplete cell debris. Pellet was resuspended in 10 ml digestion medium for 40-60 min at 37° C.

To isolate lymphocyte-enriched cell population 30 ml ice-cold serum-free RPMI 1640 was added to each tube to bring the final volume to 40 ml. The tubes were centrifuged for 10 min at 300×g (1200 rpm in IEC Centra 8R), 4° C. The supernatant was discarded and the cell pellet was resuspended to a final volume of 6 ml in ice-cold 45% Percoll in serum-free RPMI 1640, underlay with 70% Percoll in PBS to obtain a gradient.

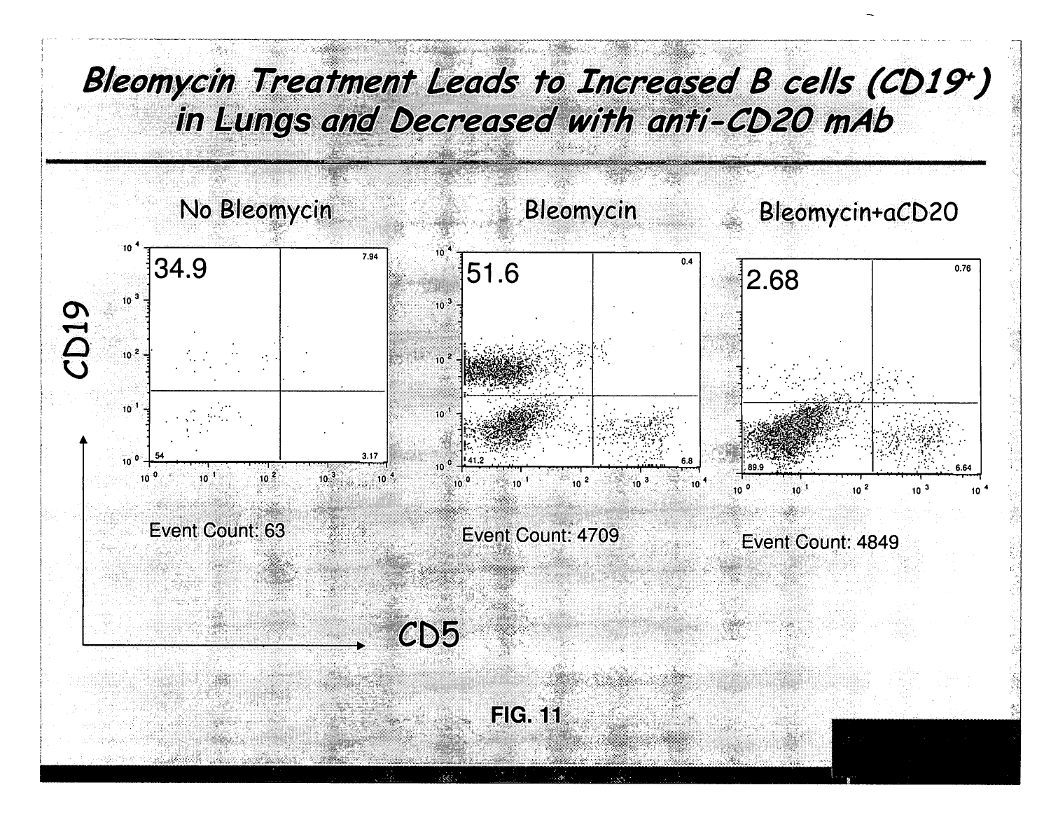

The interface was harvested ad 10 vol of ice-cold serum-free RPMI 1640 was added and the tubes centrifuged for 10 min at 400×g (1500 rpm in ]IEC Centra 8R), 4° C. CD5 and CD19 expressing cells were analyzed using FACS. (FIGS. 11, 12 and 13).

Results

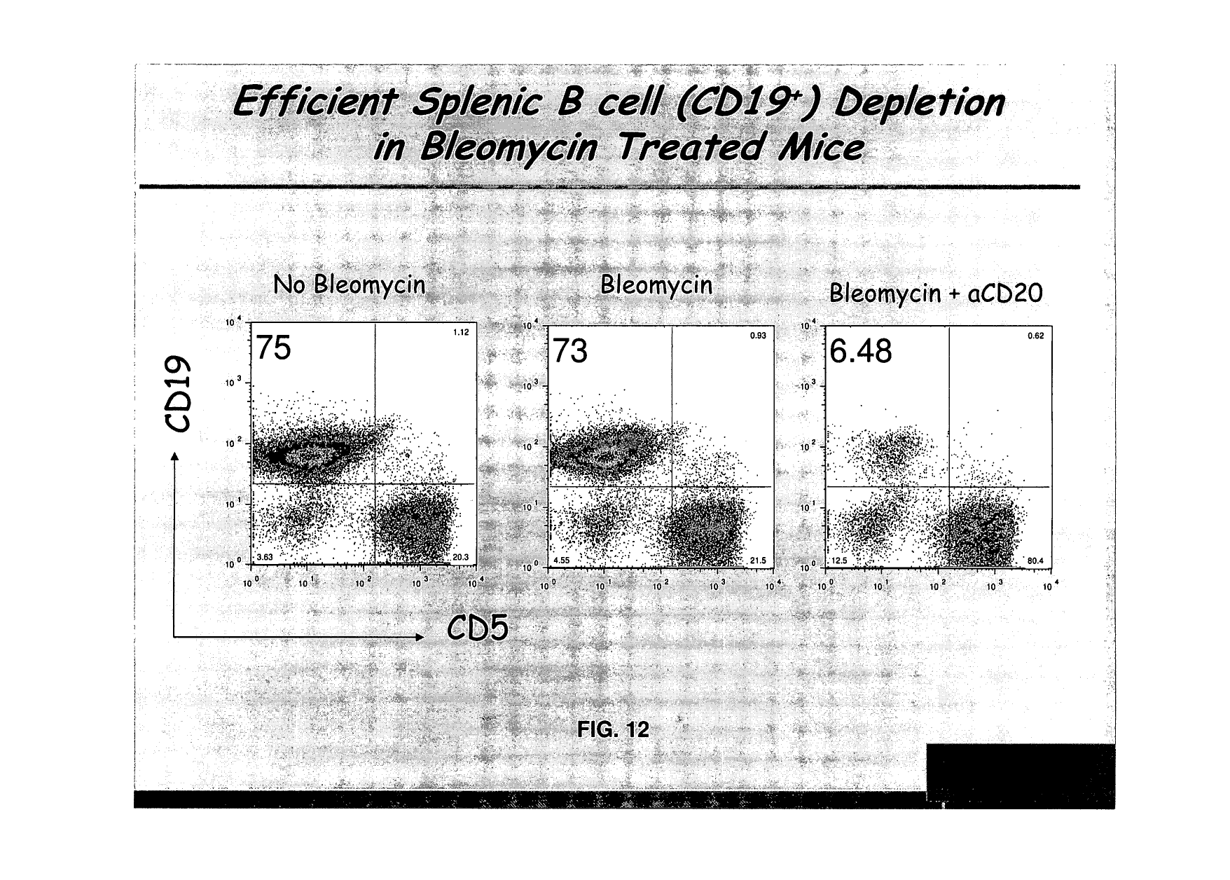

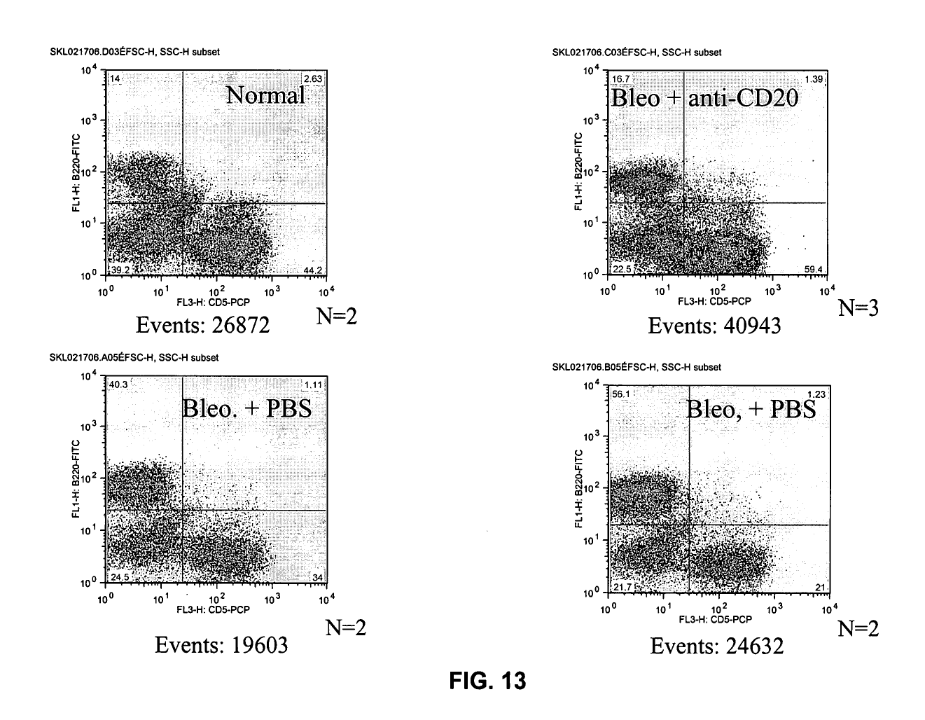

As shown in FIGS. 11 and 13, mice treated with bleomycin exhibited an increase in B-cells (CD 19⁺) in lungs, and this increase was significantly reduced in mice that received the anti-CD20 antibody in addition to bleomycin. As shown in FIG. 12, treatment with the anti-CD20 antibody also effectively depleted B-cells in the spleen. As noted in Example 2, above, pulmonary fibrosis can be induced in animal models by exposure to bleomycin. These results therefore support the conclusion that B-cell antagonists, such as an anti-CD20 antibody, are effective in treating fibrosis conditions.

### Example 5

### An Anti-CD20 Monoclonal Antibody is Protective Against CCl₄-Induced Liver Fibrosis

Introduction

As demonstrated in Example 1, liver fibrosis is induced in animal models by exposure to CCl₄, and the extent of CCl₄-induced liver fibrosis is reduced in B-cell deficient mice. In this Example, it is shown that CCl₄-induced liver fibrosis is significantly reduced in mice that are treated with an anti-CD20 antibody. These results provide yet further evidence for the use of B-cell antagonists to treat fibrosis conditions.

Materials and Methods

An anti-murine CD20 B-cell depleting antibody (designated “18B12,” developed at Biogen Idec, U.S. Appl. No. 60/741,491) was tested in a mouse model of liver fibrosis which was induced by administering the chemical carbon tetrachloride (CCl₄). A dose of 1.75 ml/Kg of CCl₄ prepared in mineral oil was administered once a week for six weeks and mice were concomitantly treated (intraperitoneally) with PBS alone, 250 μg of the anti-CD20 monoclonal antibody, or 250 μg of an isotype control monoclonal antibody. Mice were injected with PBS and antibodies one week prior to the administration of the first dose of CCl₄ and one day prior to each subsequent dose of CCl₄. Seven days after the sixth CCl₄ dose, mice were sacrificed and livers were excised and immunostained for the expression of smooth muscle actin, a marker of fibrosis.

Results

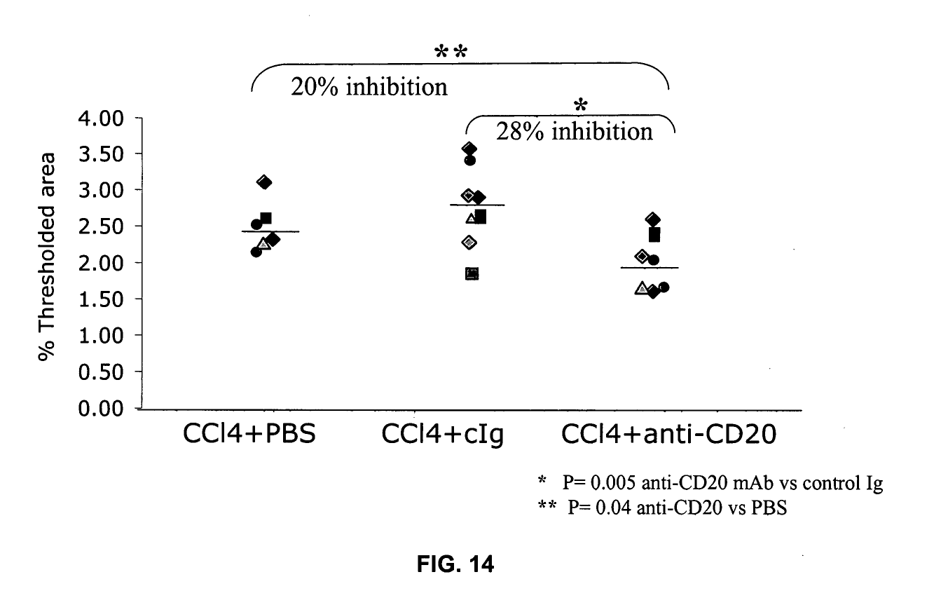

As shown in FIG. 14, the extent of liver fibrosis (as indicated by smooth muscle actin staining) in animals treated with the anti-CD20 antibody was approximately 20% less than in the control animals receiving PBS, and approximately 28% less than in animals receiving the control monoclonal antibody. Again, this Example shows the applicability of the methods of the present invention in treating, retarding, or preventing the onset or progression of fibrotic conditions, particularly in the liver.

### Example 6

### Methods of Treating Fibrosis Conditions

A patient diagnosed with one or more symptoms of a fibrosis condition is treated according to this example. Examples of fibrosis conditions to be treated herein include, e.g., lung diseases associated with injury/fibrosis, chronic nephropathies associated with injury/fibrosis (kidney fibrosis), gut fibrosis, liver fibrosis (including, e.g., cirrhosis); head and neck fibrosis, corneal scarring, vascular disorders, and autoimmune diseses associated with fibrosis such as, e.g., scleroderma, lupus, and graft-versus-host disease.

The patient is treated with rituximab or humanized 2H7, or a fragment (such as a Fab, F(ab′).sub.2, Fv, scFv or diabody) of rituximab or humanized 2H7.

Preferably, the antibody is administered intravenously (IV) to the patient according to any of the following dosing schedules:

(A) 50 mg/m² on day 1; 150 mg/m² on days 8, 15 and 22;

(B) 150 mg/m² on day 1; 375 mg/m² on days 8, 15 and 22; or

(C) 375 mg/m² on days 1, 8, 15 and 22.

The patient treated with the CD20 antibody will display an improvement in symptoms of the fibrosis condition.

In an alternative dosing regimen, the patient is treated with rituximab as set forth in schedule A immediately above and with an antibody to αᵥβ₆ as described in U.S. Pat. No. 6,316,601 which is incorporated herein in its entirety by reference. Again the patient will display an improvement in symptoms of the fibrosis condition.

In another alternative dosing regimen, the patient is treated with rituximab as set forth in Schedule B. The level of the patient's peripheral B-cells are monitored as is the amount of collagen deposition in the organ of interest. Eight months later, as the patient's reconstituted B-cell immune response and/or amount of collagen deposition reaches a predetermined level, the patient is re-treated with rituximab according to Schedule A.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method for treating a fibrosis condition, said method comprising administering to a patient in need of such treatment a therapeutically effective amount of a B-cell antagonist.

2. The method of claim 1, wherein said B-cell antagonist is an antibody against a B-cell surface antigen.

3. The method of claim 2, wherein said B-cell surface antigen is CD20.

4. The method of claim 2, wherein said B-cell surface antigen is selected from the group consisting of CD10, CD19, CD20, CD21, CD22, CD23, CD24, CD37, CD40, CD52, CD53, CD72, CD73, CD74, CDw75, CDw76, CD77, CDw78, CD79a, CD79b, CD80, CD81, CD82, CD83, CDw84, CD85, CD86, TLR-7, TLR-9, CXCR3, APRIL, BR3, BCMA and TACI.

5. The method of claim 2, wherein said antibody is a monoclonal antibody.

6. The method of claim 2, wherein said antibody is a monoclonal antibody against CD20.

7. The method of claim 6, wherein said antibody is a chimeric murine/human monoclonal antibody against CD20.

8. The method of claim 7, wherein said monoclonal antibody against CD20 is rituximab (RITUXAN®).

9. The method of claim 2, wherein said antibody is a humanized antibody.

10. The method of claim 2 wherein said antibody is a fully human antibody.

11. The method of claim 1, further comprising administering a therapeutically effective amount of a BAFF antagonist to said patient.

12. The method of claim 11, wherein said BAFF antagonist is a polypeptide comprising an amino acid sequence selected from the group consisting of ECFDLLVRAWVPCSVLK (SEQ ID NO:15), ECFDLLVRHWVPCGLLR (SEQ ID NO:16), ECFDLLVRRWVPCEMLG (SEQ ID NO:17), ECFDLLVRSWVPCHMLR (SEQ ID NO:18), and ECFDLLVRHWVACGLLR (SEQ ID NO:19).

13. The method of claim 11 wherein said BAFF antagonist comprises a soluble fusion protein comprising at least a portion of a BAFF receptor and a portion of a constant region of an immunoglobulin.

14. The method of claim 1, wherein the patient does not have an autoimmune disorder.

15. The method of claim 1, wherein the patient is not at risk of having an autoimmune disorder.

16. The method of claim 1, wherein said B-cell antagonist causes a 20% depletion of peripheral B-cells in said patient within 24 hours of administration of said B-cell antagonist to said patient.

17. The method of claim 1, wherein said B-cell antagonist causes a 60% depletion of peripheral B-cells in said patient within 24 hours of administration of said B-cell antagonist to said patient.

18. The method of claim 1, wherein said B-cell antagonist causes an 80% depletion of peripheral B-cells in said patient within 24 hours of administration of said B-cell antagonist to said patient.

19. A method for treating pulmonary fibrosis, said method comprising administering to a patient in need of such treatment a therapeutically effective amount of a B-cell antagonist.

20. The method of claim 19, wherein said B-cell antagonist is an antibody against CD20.

21. The method of claim 20, wherein said antibody is a chimeric murine/human monoclonal antibody against CD20.

22. The method of claim 21, wherein said antibody against CD20 is rituximab (RITUXAN®).

23. The method of claim 1, wherein after said B-cell antagonist is administered to said patient, said patient exhibits a decrease in one or more markers of fibrosis as compared to said patient prior to administration of said B-cell antagonist.

24. The method of claim 23, wherein said one or more markers of fibrosis is smooth muscle actin deposition or collagen deposition.

25. The method of claim 24, wherein after said B-cell antagonist is administered to said patient, the extent of smooth muscle actin staining observed on one or more tissues in said patient is at least 5% less than the extent of smooth muscle actin staining observed on said one or more tissues in said patient prior to administration of said B-cell antagonist.

26. The method of claim 24, wherein after said B-cell antagonist is administered to said patient, the extent of smooth muscle actin staining observed on one or more tissues in said patient is at least 25% less than the extent of smooth muscle actin staining observed on said one or more tissues in said patient prior to administration of said B-cell antagonist.

27. The method of claim 24, wherein after said B-cell antagonist is administered to said patient, the extent of smooth muscle actin staining observed on one or more tissues in said patient is at least 50% less than the extent of smooth muscle actin staining observed on said one or more tissues in said patient prior to administration of said B-cell antagonist.

28. The method of claim 24, wherein after said B-cell antagonist is administered to said patient, the extent of collagen staining observed on one or more tissues in said patient is at least 5% less than the extent of collagen staining observed on said one or more tissues in said patient prior to administration of said B-cell antagonist.

29. The method of claim 24, wherein after said B-cell antagonist is administered to said patient, the extent of collagen staining observed on one or more tissues in said patient is at least 25% less than the extent of collagen staining observed on said one or more tissues in said patient prior to administration of said B-cell antagonist.

30. The method of claim 24, wherein after said B-cell antagonist is administered to said patient, the extent of collagen staining observed on one or more tissues in said patient is at least 50% less than the extent of collagen staining observed on said one or more tissues in said patient prior to administration of said B-cell antagonist.

31. A method for treating hepatic fibrosis, said method comprising administering to a patient in need of such treatment a therapeutically effective amount of a B-cell antagonist.

32. The method of claim 31, wherein said B-cell antagonist is an antibody against CD20.

33. The method of claim 32, wherein said antibody is a chimeric murine/human monoclonal antibody against CD20.

34. The method of claim 33, wherein said antibody against CD20 is rituximab (RITUXAN®.

35. A method for treating renal fibrosis, said method comprising administering to a patient in need of such treatment a therapeutically effective amount of a B-cell antagonist.

36. The method of claim 35, wherein said B-cell antagonist is an antibody against CD20.

37. The method of claim 36, wherein said antibody is a chimeric murine/human monoclonal antibody against CD20.

38. The method of claim 37, wherein said antibody against CD20 is rituximab (RITUXAN®).

39. A method for treating a fibrosis condition, said method comprising administering to a patient in need of such treatment a therapeutically effective amount of a B-cell antagonist and a therapeutically effective amount of an integrin receptor antagonist.

40. The method of claim 39, wherein said integrin receptor antagonist is an antibody specific for an integrin receptor.

41. The method of claim 40, wherein said integrin receptor is selected from the group consisting of αvβ6, αvβ5, α5β1, α4β1, α4β1, and α4β7.

42. The method of claim 41, wherein said integrin receptor is an α4β1 or an α4β7 integrin receptor.

43. The method of claim 42, wherein said integrin receptor antagonist is natalizumab (TYSABRI®).

44. The method of claim 39, wherein the patient does not have an autoimmune disorder.

45. The method of claim 39, wherein the patient is not at risk of having an autoimmune disorder.

46. A method for treating a fibrosis condition, said method comprising administering to a patient in need of such treatment a therapeutically effective amount of rituximab (RITUXAN®) and a therapeutically effective amount of natalizumab (TYSABRI®).

47. A method for preventing a fibrosis condition, said method comprising administering to a patient at risk of developing one or more fibrosis conditions a therapeutically effective amount of a B-cell antagonist.

48. The method of claim 47, wherein said patient at risk of developing one or more fibrosis conditions has been exposed to one or more environmental conditions that are known to increase the risk of lung, liver or kidney fibrosis.


SEQUENCE LISTING (20 sequences)

SEQ ID NO: 1 - protein, 232 aa, Homo sapiens
MGWSCIILFLVATATGVHSDIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 471 aa, Homo sapiens
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIERTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 471 aa, Homo sapiens
MGWSCIILFLVATATGVHSEVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 107 aa, Homo sapiens
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKR

SEQ ID NO: 5 - protein, 122 aa, Homo sapiens [sequence omitted]

SEQ ID NO: 6 - protein, 213 aa, Homo sapiens
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 452 aa, Homo sapiens
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 452 aa, Homo sapiens
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 121 aa, Homo sapiens
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVVYYSNSYWYFDVWGTGTTVTVS

SEQ ID NO: 10 - protein, 106 aa, Homo sapiens [sequence omitted]

SEQ ID NO: 11 - protein, 107 aa, Homo sapiens
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKR

SEQ ID NO: 12 - protein, 122 aa, Homo sapiens
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSS

SEQ ID NO: 13 - protein, 451 aa, Homo sapiens
EVQLVESGGGLVQPGGSLRLSCAASGYTFTSYNMHWVRQAPGKGLEWVGAIYPGNGATSYNQKFKGRFTISVDKSKNTLYLQMNSLRAEDTAVYYCARVVYYSYRYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNATYRVVSVLTVLHQDWLNGKEYKCKVSNAALPAPIAATISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 291 aa, Homo sapiens
MLPGCKWDLLIKQWVCDPLGSGSATGGSGSTASSGSGSATHMLPGCKWDLLIKQWVCDPLGGGGGVDKTHTCPPCPAPELLGGPSVFLFPPKPKDTLMISRTPEVTCWWDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 17 aa, Homo sapiens
ECFDLLVRAWVPCSVLK

SEQ ID NO: 16 - protein, 17 aa, Homo sapiens
ECFDLLVRHWVPCGLLR

SEQ ID NO: 17 - protein, 17 aa, Homo sapiens
ECFDLLVRRWVPCEMLG

SEQ ID NO: 18 - protein, 17 aa, Homo sapiens
ECFDLLVRSWVPCHMLR

SEQ ID NO: 19 - protein, 17 aa, Homo sapiens [sequence omitted]

SEQ ID NO: 20 - protein, 213 aa, Homo sapiens
DIQMTQSPSSLSASVGDRVTITCRASSSVSYLHWYQQKPGKAPKPLIYAPSNLASGVPSRFSGSGSGTDFTLTISSLQPEDFATYYCQQWAFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC